(19) United States
(12) Patent Application Publication
(10) Pub. No.: US 2026/0199489 A1
(43) Pub. Date: Jul. 16, 2026

(54) STABILIZED CARBONIC ACID ADDUCTS

(71) Applicant: INFLAMED PHARMA GMBH, Jena (DE)

(72) Inventors: Beatrice ENGERT, Jena (DE); Christina TOEPEL, Jena (DE)

(73) Assignee: INFLAMED PHARMA GMBH, Jena (DE)

(21) Appl. No.: 19/134,412

(22) PCT Filed: Dec. 1, 2023
(86) PCT No.: PCT/EP2023/083916

§ 371 Date: Jul. 28, 2025

(30) Foreign Application Priority Data
Dec. 2, 2022 (EP) 22210976.1

(51) Int. Cl. A61K 47/58; A61K 47/52

(52) U.S. Cl. CPC A61K 47/58; A61K 47/52

(57) ABSTRACT

The invention relates to stabilized carbonic acid adducts (SCAA1), (SCAA2), (SCAA3) and (SCAA4) which comprise carbonic acid, at least one amine (AM), optionally, at least one salt (S), and polyvinylpyrrolidone.

### TECHNICAL FIELD OF THE INVENTION

The invention relates to stabilized carbonic acid adducts (SCAA1), (SCAA2), (SCAA3) and (SCAA4) which comprise carbonic acid, at least one amine (AM), optionally, at least one salt (S), and polyvinylpyrrolidone.

### BACKGROUND ART

WO2006/007835 A2 and WO2019048590 A2 disclose carbonic acid adducts of amines, which are of interest in particular for pharmaceutical-medical applications. It is known from this disclosure, in particular for the carbonic acid adduct of procaine, that the transport of the active ingredient, the bioavailability and the tolerability, is improved, inter alia also in the case of acidoses. Likewise, a lack of hemolysis is observed compared to procaine. The reason for this is the presence of an acid-base pair of amine and carbonic acid, which has a high buffer capacity and is readily soluble. It is also explained that it is important for the stability of procainium bicarbonate to keep the concentration of basic substances, such as procaine or carbonate, low, for example, in infusion solutions thereof. The basicity of the carbonate promotes saponification of procaine. In addition, even small amounts of carbonate catalyze the decomposition of procainium bicarbonate into procaine and diprocainium carbonate. One possibility for stabilizing procainium bicarbonate or lidocainium bicarbonate is incorporation into mineral salts such as sodium chloride and/or dextrans, starch or cellulose to form clathrates or clusters. Furthermore, the addition of CO₂ is beneficial, which lowers the pH value and brings the proportion of procainium bicarbonate to almost 100%.

WO2019048590 A2 additionally discloses that the storage stability of the carbonic acid adducts can be increased by suitable process steps, inter alia by optimizing the CO₂ concentration in the solution during production.

However, there is a further need for further formulations of carbonic acid adducts which have increased stability, are easier to formulate, facilitate general handling and/or enable multi-dispensing systems.

### SUMMARY OF THE INVENTION

The invention relates to a stabilized carbonic acid adduct (SCAA1) comprising carbonic acid, at least one amine (AM), and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
d) freezing the solution yielded after completion of step c),
e) storing the solution frozen in step d) at −100 to 0° C. for no longer than 4 days,
g1) adding polyvinylpyrrolidone to the carbonic acid adduct (CAA) yielded in the preceding steps to yield the stabilized carbonic acid adduct (SCAA1),
wherein the content of CO₂ in the solution which is subjected to step d) is at least 6 g/l, preferably at least 7 g/l, more preferably at least 8 g/l, particularly preferably 8.1 g/l to 9.5 g/l, and the amine (AM) can also be employed in the form of a salt.

The stabilized carbonic acid adduct (SCAA2) comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
g2) adding polyvinylpyrrolidone to the solution (B) yielded in step c) to yield the stabilized carbonic acid adduct (SCAA2).
wherein the content of CO₂ in the solution (B) which is subjected to step c) is at least 10 g/l, preferably at least 12 g/l, more preferably at least 14 g/l, and most preferably at least 14 to 16 g/l, and the amine (AM) can also be employed in the form of a salt.

The stabilized carbonic acid adduct (SCAA3) comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
g3) adding polyvinylpyrrolidone to the solution (B) yielded in step c) to yield the solution (C),
h) freezing the solution yielded after completion of step g3),
j) storing the solution frozen in step h) at −100 to 0° C. for no longer than 4 days,
wherein the content of CO₂ in the solution which is subjected to step h) is at least 7 g/l, preferably at least 7.5 g/l, and the amine (AM) can also be employed in the form of a salt,
wherein the steps are carried out in the order listed above: a), optionally b), c), g3), h) and j).

The invention also relates to a stabilized carbonic acid adduct (SCAA4) comprising carbonic acid, at least one amine (AM), and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
d) freezing the solution yielded after completion of step c),
e) storing the solution frozen in step d) at −100 to 0° C. for no longer than 4 days,
f) drying the solution stored in step e) to yield the carbonic acid adduct (CAA),
k) drying a solution saturated with CO₂ comprising the carbonic acid adduct (CAA) from step f), and polyvinylpyrrolidone,
wherein the content of CO₂ in the solution which is subjected to step d) is at least 6 g/l, preferably at least 7 g/l, more preferably at least 8 g/l, particularly preferably 8.1 g/l to 9.5 g/l, and the amine (AM) can also be employed in the form of a salt.

Furthermore, the invention relates to the stabilized carbonic acid adducts (SCAA1), (SCAA2), (SCAA3) and (SCAA4) for use in a method of anesthesia, of analgesia, of concomitant treatment of cancer, of anti-inflammatory treatment, of treatment of amyotrophic lateral sclerosis, of supporting wound healing, in particular in burns, open wounds and scars, of treatment of neurogenic inflammations such as multiple sclerosis, MMN (multifocal motor neuropathy), of treatment of sinusitis, of treatment of asthma, of treatment of rheumatoid arthritis, of treatment of Alzheimer's disease, of treatment of dementia, of supporting convalescence and of supporting anti-aging, of treatment of burnout syndrome, of treatment of osteoarthritis, of treatment of polyarthritis, of treatment of pain syndrome and general pain, of pre- and post-operative treatment (also for bone fractures), of preventive and rehabilitation medicine, of treatment of zoster neuralgia, of accompanying treatment of zoster neuralgia, of treatment of diseases of the abdominal organs such as liver, gallbladder, pancreas, intestines, of treatment of gastrointestinal diseases (ulcerative colitis, Crohn's disease), of treatment of ankylosing spondylitis, of treatment of chronic musculoskeletal pain, of treatment of diabetes (improvement of blood sugar levels), of reducing edema, of treatment of fatigue, long-COVID, of improving microcirculation, of comedication to opioids.

The stabilized carbonic acid adducts (SCAA1), (SCAA2), (SCAA3) and (SCAA4) enable in one embodiment a multi-dispensing system, e.g., for neural therapeutic purposes. Such an application was not previously possible with the prior art carbonic acid adducts described above. A multi-dispensing system means that several doses can be dispensed from one container. The stabilized carbonic acid adduct (SCAA2) for buccal applications is stable for about 7 days when applied daily and stored in the refrigerator. In another embodiment as a drinking solution, the stabilized carbonic acid adduct (SCAA2) is stable for 24 h at room temperature and for 10 months when stored in a closed container in the refrigerator.

The stabilized carbonic acid adducts (SCAA3) and (SCAA4) prove to be quite stable and can, in one embodiment, be pressed directly into tablets as a solid mixture. Alternatively, ointments with water-containing bases can be produced from the stabilized carbonic acid adduct (SCAA3). Compared to the known carbonic acid adducts from the prior art, the stabilized carbonic acid adduct (SCAA3) has a delayed release.

### BRIEF DESCRIPTION OF THE FIGURES

FIG. 1: Stability of SCAA1 (CAA:PVP 1:3 (w/w)) when stored in the refrigerator, based on procaine.

FIG. 2: Stability of SCAA1 (CAA:PVP 1:3 (w/w)) when stored at room temperature, based on procaine

FIG. 3: Stability of SCAA3 and SCAA4 (with PVP30), based on procaine. The production of SCAA3 is carried out as described in the production example for SCAA3.

FIG. 4: Stability of SCAA2 (CAA:PVP 1:5 (w/w)) when stored in the refrigerator (multi-dispensing system), based on procaine

FIG. 5: Stability of SCAA3 and SCAA4 (with PVP17 and galactose), each based on procaine. The production of SCAA3 is carried out as described in the production example for SCAA3.

FIG. 6: Stability of SCAA1 (CAA:PVP 1:3 (w/w)) when stored in the refrigerator (multi-dispensing system), based on procaine.

FIG. 7: Stability of SCAA3 and SCAA4 (with PVP17 and galactose) when stored in the refrigerator (multi-dispensing system), each based on procaine. The production of SCAA3 is carried out as in the production example for SCAA3.

FIG. 8: Stability of SCAA4 tablets (without further additives), based on procaine.

FIG. 9: X-ray diffraction diagram (XRD): Wherein 1 is the curve for SCAA3 (plate lyophilization), 2 is the curve for SCAA3 (plate lyophilization, scaled up), 3 is the curve for SCAA3 (flask lyophilization), 4 is the curve for carbonic acid adduct CAA without PVP addition (production of CAA is carried out as described by way of example in the experimental part in WO2019048590 A2), all based on procaine. The production of SCAA3 is carried out as described in the production example for SCAA3.

FIG. 10: Infrared spectrogram: Wherein a is SCAA3 (flask lyophilization, based on procaine, b is carbonic acid adduct CAA without PVP addition (production of CAA is carried out as described by way of example in the experimental part in WO2019048590 A2, based on procaine), c is SCAA1 (mixture of CAA and PVP 1:3 (w/w)).

FIG. 11: Octanol measurements: The octanol-water partition coefficient was determined as a measure of membrane permeability. Procluster stable corresponds to SCAA3 based on procaine. Procluster corresponds to the carbonic acid adduct CAA without PVP addition. The production of CAA is carried out as described by way of example in the experimental part of WO2019048590 A2. It turns out that SCAA3 has a higher membrane permeability than the carbonic acid adduct CAA without PVP addition and both have a significantly higher membrane permeability than procaine hydrochloride.

FIG. 12: Stability studies by means of infrared spectroscopy: A: I corresponds to carbonic acid adduct CAA based on procaine stored at room temperature. The production of CAA is carried out as described by way of example in the experimental part of WO2019048590 A2. II corresponds to carbonic acid adduct CAA based on procaine at the beginning of the storage period. It turns out that the carbonic acid adduct CAA is only stable for 3 to 6 months when stored at room temperature and after this time the decomposition product, free procaine, can be detected in the infrared spectrum. B: III corresponds to the stabilized carbonic acid adduct SCAA3 based on procaine at the beginning of the storage period at room temperature. IV corresponds to the stabilized carbonic acid adduct SCAA3 based on procaine after 18 months of storage at room temperature. The stabilized carbonic acid adduct SCAA3 remains stable even after 18 months of storage, as evidenced by the infrared spectrum IV in which no procaine band appears. The production of SCAA3 is carried out as described in the production example for SCAA3.

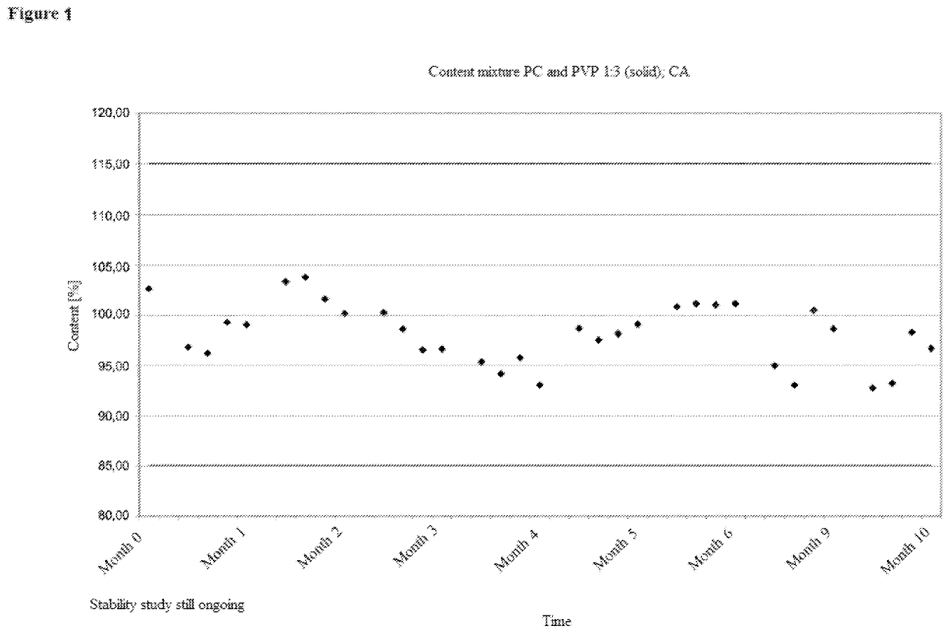

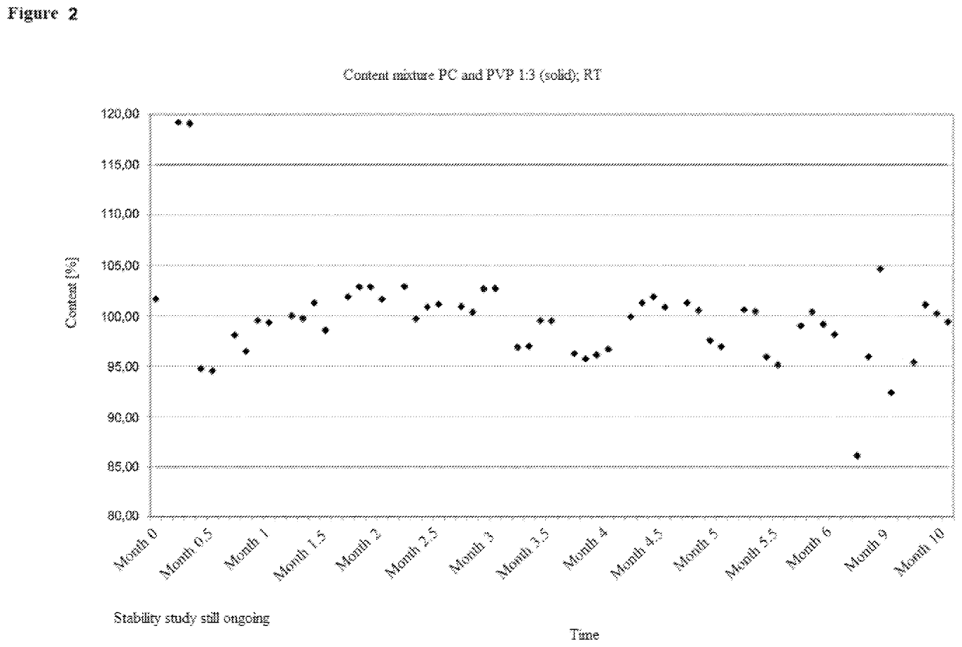

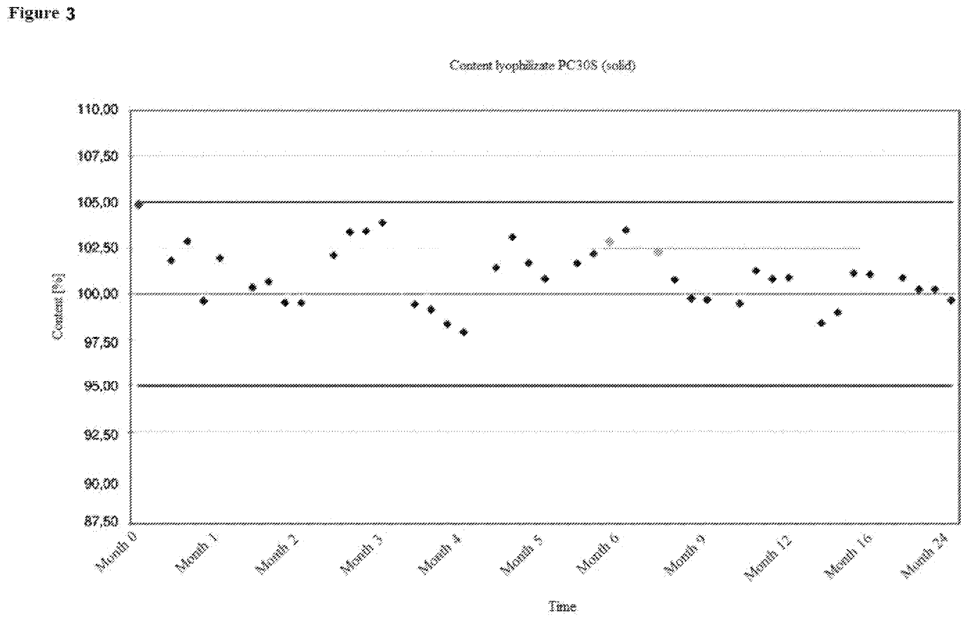

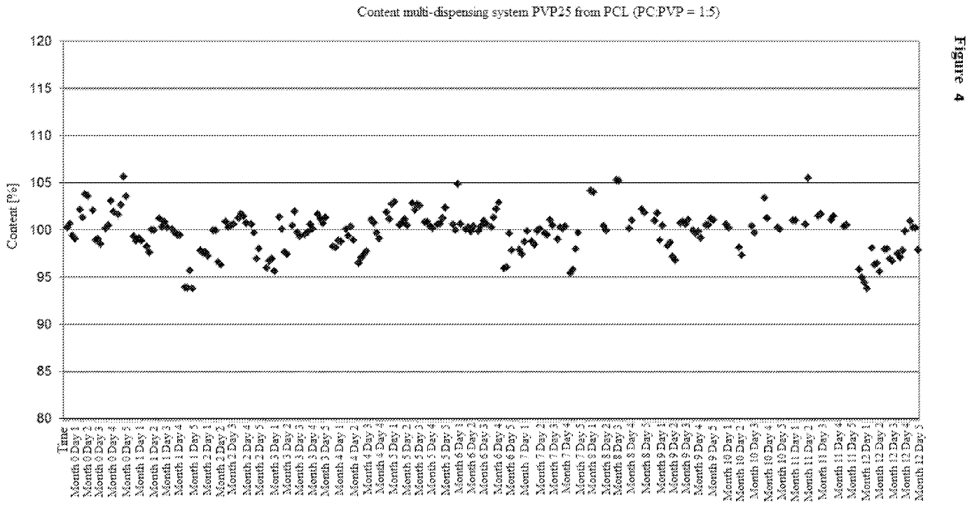

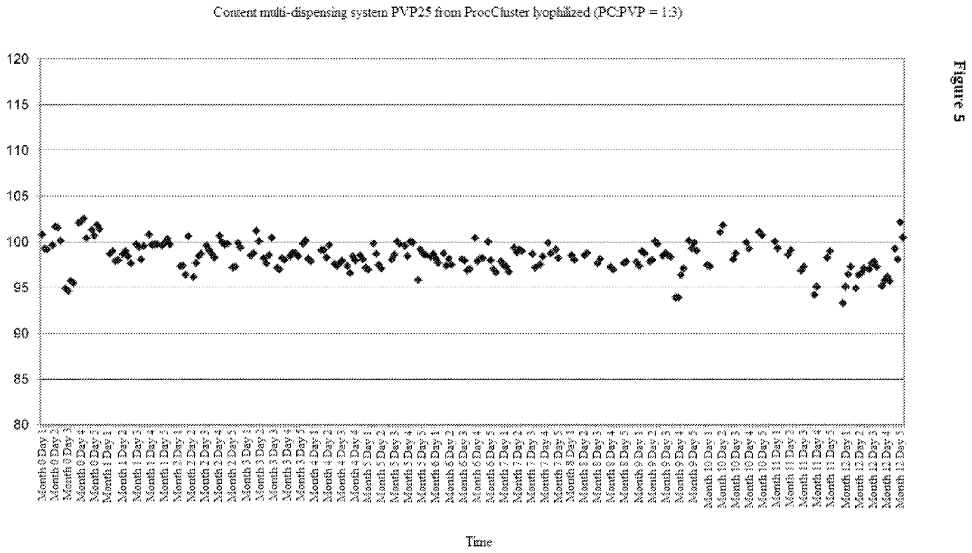

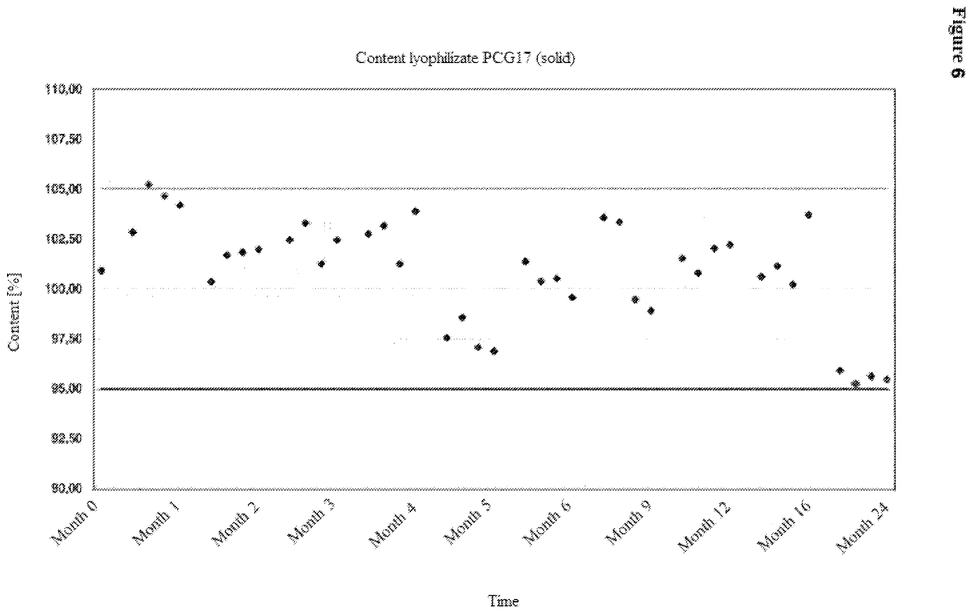

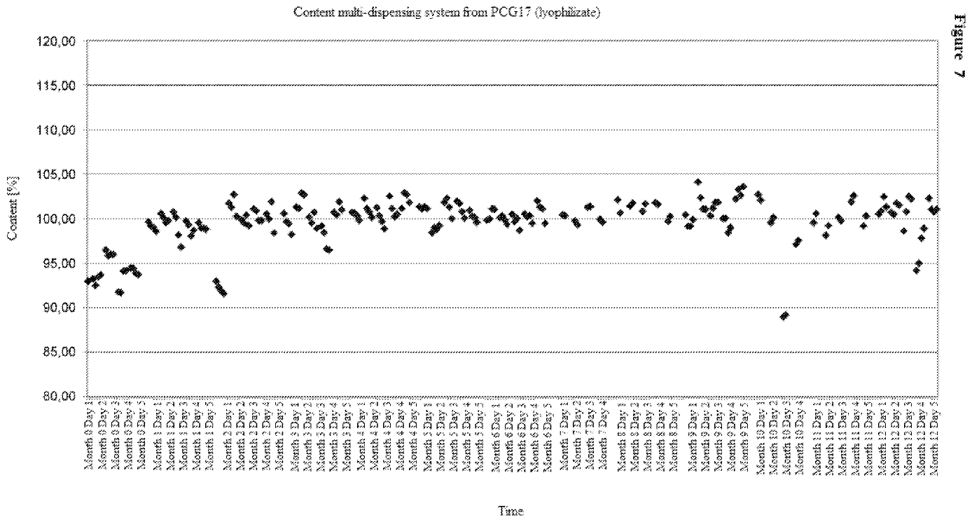

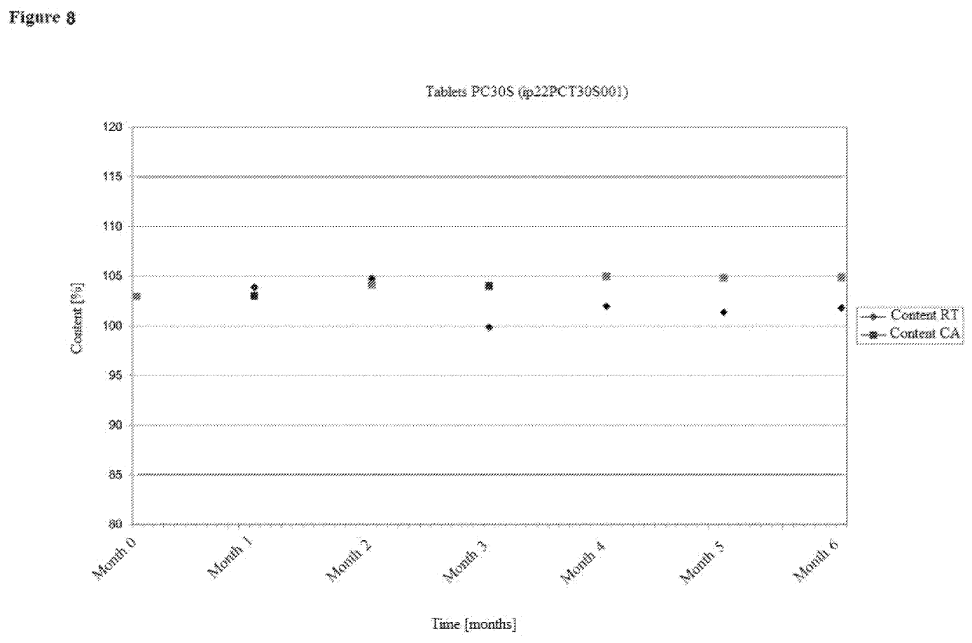

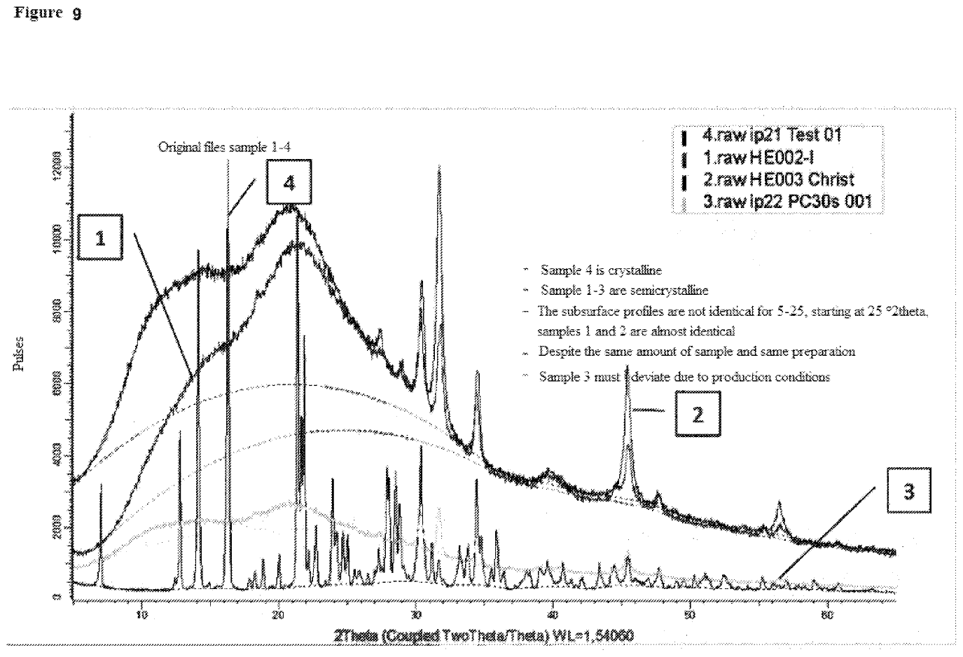

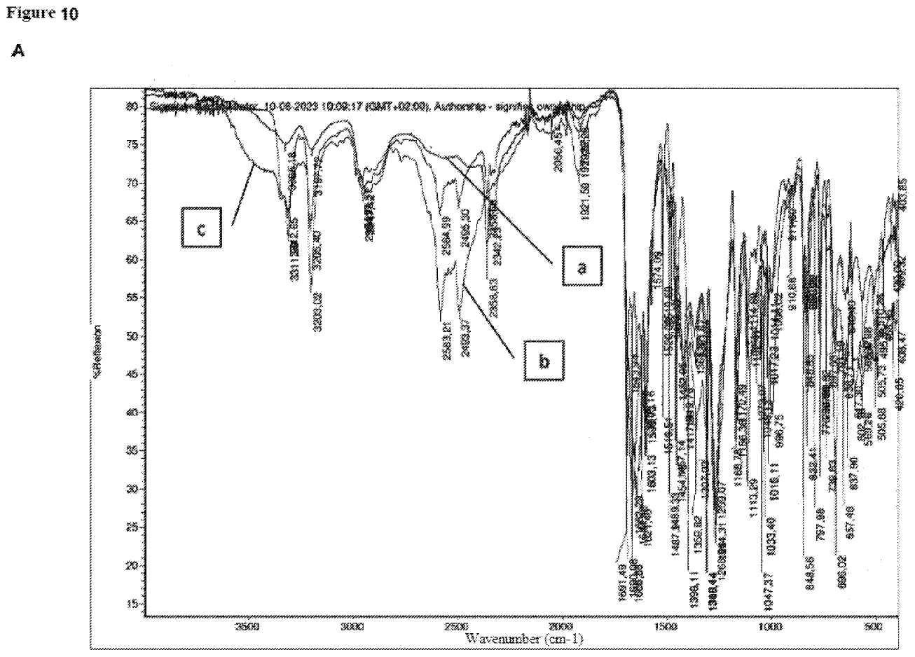

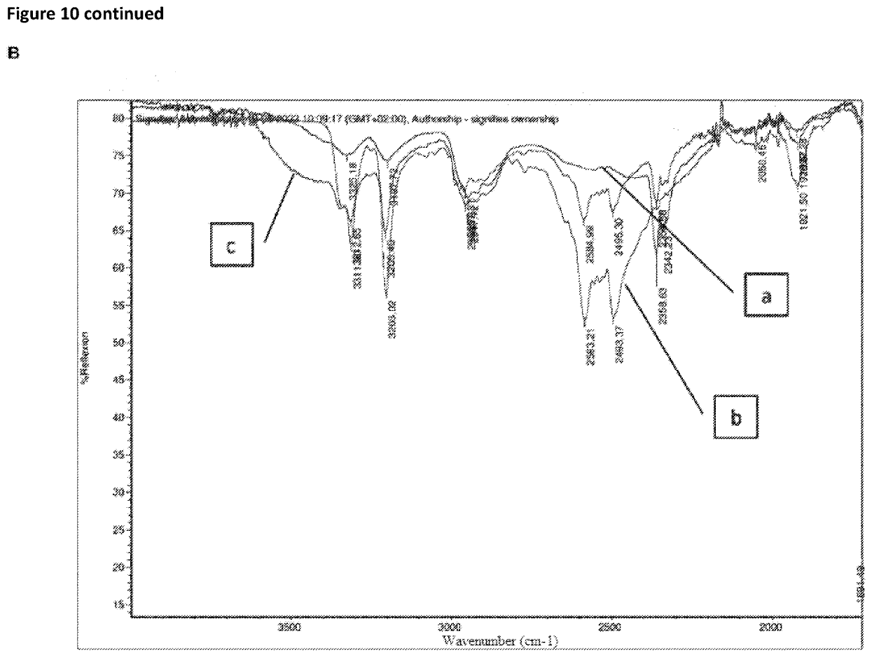

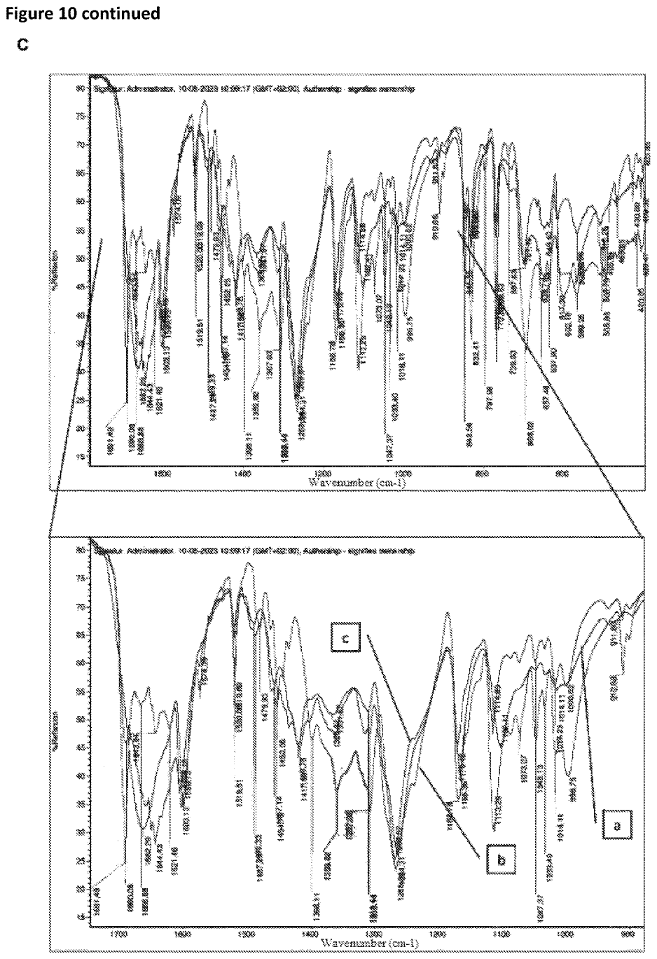

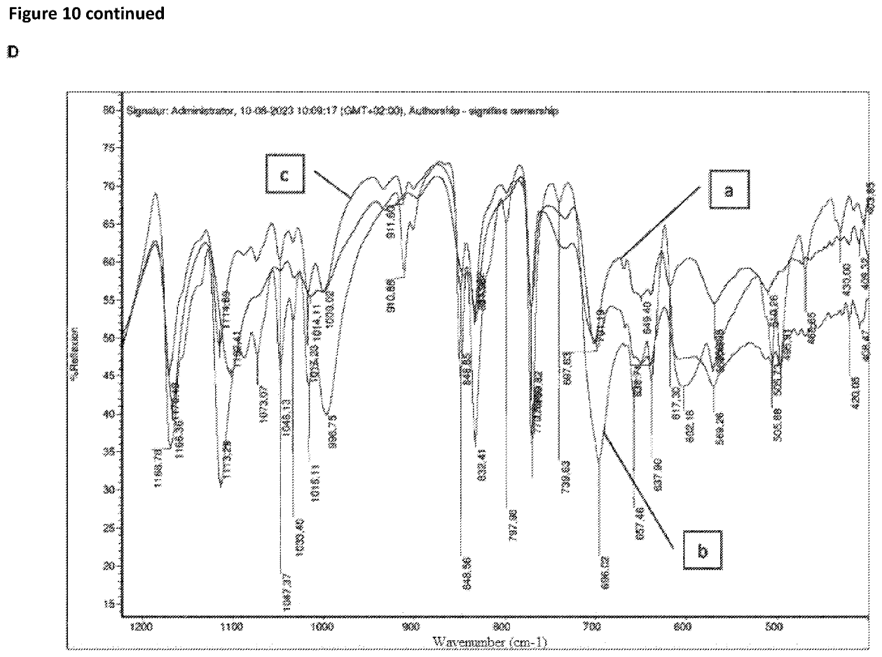

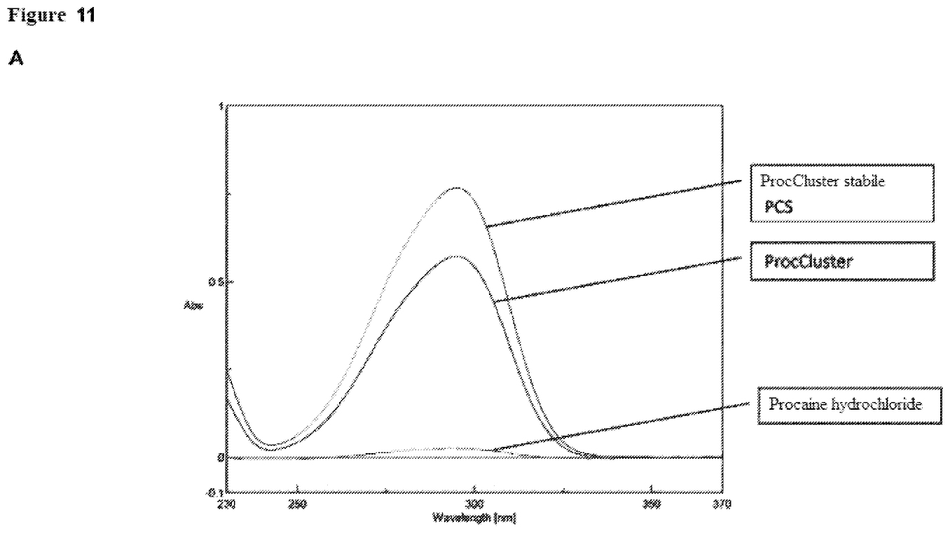

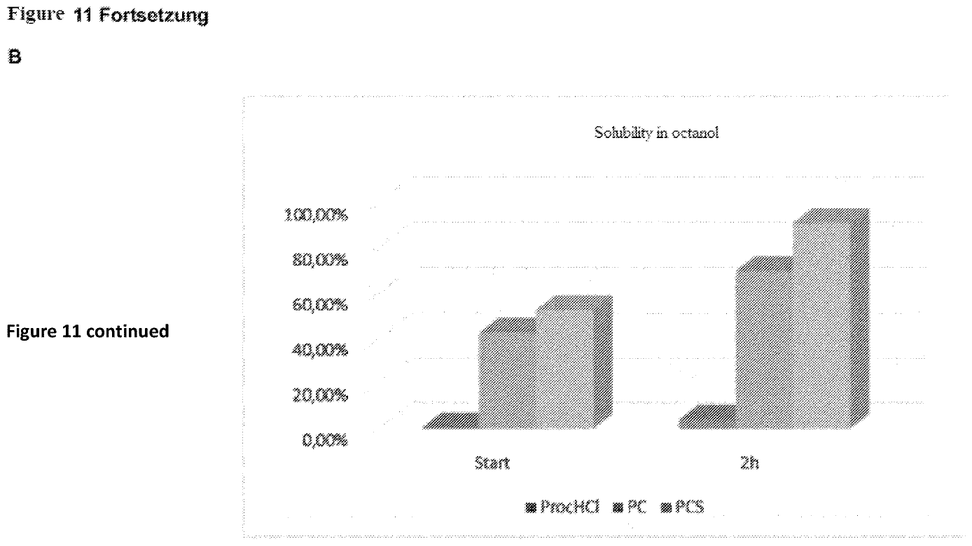

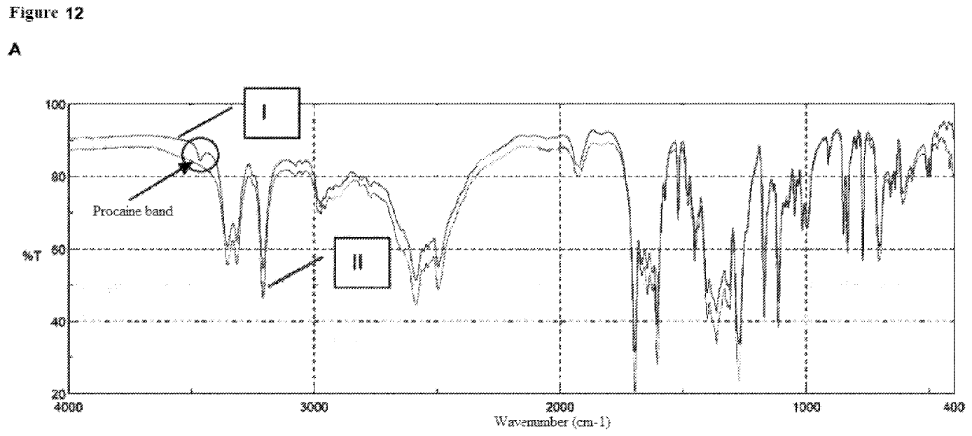

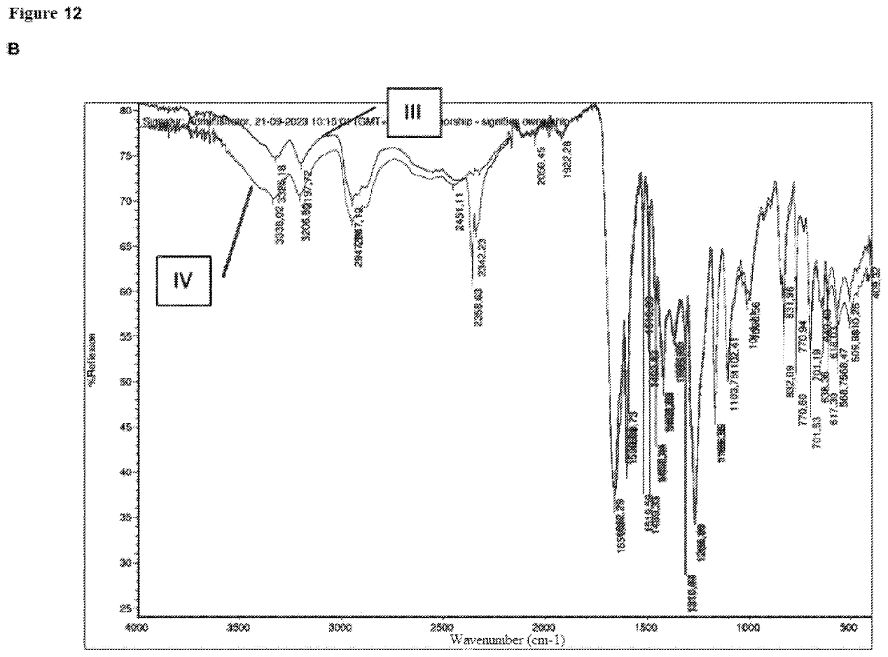

### DETAILED DESCRIPTION OF THE INVENTION

### Substances

The carbonic acid adducts (SCAA1), (SCAA2), (SCAA3) and (SCAA4) each comprise carbonic acid, at least one amine (AM), and, optionally, at least one salt (S).

In the context of this invention, an adduct is understood to mean a molecule which is formed by joining, based on their molecular weight, smaller molecules than the adduct via the formation of covalent or non-covalent bonds, preferably non-covalent bonds. Preferably, the adduct also comprises, as non-covalent bonds, ionic bonds between at least a portion of the particles that have joined to form the adduct.

Carbonic acid H₂CO₃ can be present in the carbonic acid adducts (SCAA1), (SCAA2), (SCAA3) and (SCAA4) in non-ionized form, in partially ionized form or even completely ionized. These different degrees of ionization of carbonic acid can also exist side by side in the carbonic acid adduct. The ionization of carbonic acid can occur by transferring one or both protons of the carbonic acid to another molecule, preferably at least one proton to the amine (AM).

The at least one amine (AM) is understood to mean a molecule that has at least one amino group, preferably one amino group. The amino group can be a primary, secondary or tertiary amino group.

The at least one amino group of the amine (AM) can be present in the carbonic acid adduct (CAA), (SCAA1), (SCAA2), (SCAA3) or (SCAA4) both in neutral form and in protonated form. If the amine (AM) has more than one amino group, all amino groups can be in neutral form, all amino groups can be in protonated form, or some of the amino groups can be in neutral form and some of the amino groups can be in protonated form. Preferably, at least one amino group is in protonated form.

Preferably, the carbonic acid adduct comprises at least one amine (AM) according to any one of the following formulas (I) and/or (II).

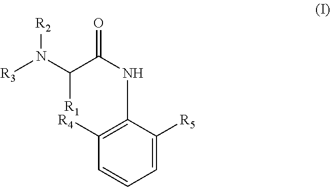

wherein in formula (I)
R₁ is H, C₁₋₁₀ alkyl, C₂₋₁₀ alkenyl, aryl or heteroaryl, preferably H or C₁₋₁₀ alkyl, more preferably H or C₁₋₄ alkyl;
R₂ is H, C₁₋₁₀ alkyl, C₂₋₁₀ alkenyl, aryl or heteroaryl, preferably H or C₁₋₁₀ alkyl, more preferably H or C₁₋₄ alkyl;
R₃ is H, C₁₋₁₀ alkyl, C₂₋₁₀ alkenyl, aryl or heteroaryl, preferably H or C₁₋₁₀ alkyl, more preferably H or C₁₋₄ alkyl;
wherein, optionally, R₁ and R₃ may be linked together, and together with the nitrogen atom bonded to R₃ and the carbon atom bonded to R₁ may form a saturated or unsaturated, preferably a saturated, ring. Preferably, the ring is a 4, 5 or 6 membered ring, more preferably a 5 or 6 membered ring;
R₄ is H, halo, C₁₋₁₀ alkyl, C₂₋₁₀ alkenyl, aryl, heteroaryl, preferably H or methyl;
R₅ is H, halo, C₁₋₁₀ alkyl, C₂₋₁₀ alkenyl, aryl or heteroaryl, preferably H, methyl or halo;

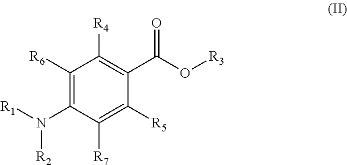

wherein in formula (II)
R₁ is H, C₁₋₁₀ alkyl, C₂₋₁₀ alkenyl, aryl or heteroaryl, preferably H or C₁₋₁₀ alkyl, more preferably H;
R₂ is H, C₁₋₁₀ alkyl, C₂₋₁₀ alkenyl, aryl or heteroaryl, preferably H or C₁₋₁₀ alkyl, more preferably H;
R₃ is H, C₁₋₁₀ alkyl, C₂₋₁₀ alkenyl, aryl, heteroaryl or —(CH₂)ₙNR₈R₉, preferably —(CH₂)ₙNR₈R₉;
n is 1 to 5, preferably 1 to 3, more preferably 1 to 2;
R₈ is C₁₋₁₀ alkyl, preferably C₁₋₂ alkyl;
R₉ is C₁₋₁₀ alkyl, preferably C₁₋₂ alkyl;
R₄ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or halo;
R₅ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or halo;
R₆ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or —O—C₁₋₁₀ alkyl;
R₇ is H, halo, C₁₋₁₀ alkyl, C₂₋₁₀ alkenyl, aryl or heteroaryl, or —O—C₁₋₁₀ alkyl, preferably H, halo, C₁₋₁₀ alkyl or —O—C₁₋₁₀ alkyl, more preferably H or —O—C₁₋₁₀ alkyl.

In the present invention, definitions such as C₁₋₁₀ alkyl, as defined for example for the radical R₁ of formula (I), mean that this substituent (radical) is a saturated alkyl radical having a carbon number of 1 to 10. The alkyl radical can be linear or branched and, if appropriate, cyclic. Alkyl radicals that have both a cyclic and a linear component are likewise included in this definition. The same applies to other alkyl radicals such as a C₁₋₂ alkyl radical. If appropriate, alkyl radicals can also be mono- or polysubstituted with functional groups such as amino, hydroxy, halo, aryl or heteroaryl. Unless otherwise stated, alkyl radicals preferably do not have any functional groups as substituents. Examples of alkyl radicals are methyl, ethyl, n-propyl, n-butyl, n-pentyl, n-hexyl, n-heptyl, n-octyl, n-nonyl, iso-propyl (also called 2-propyl or 1-methylethyl), iso-butyl, tert-butyl, sec-pentyl, neo-pentyl, 1,2-dimethylpropyl, iso-amyl, iso-hexyl, iso-heptyl.

In the present invention, definitions such as C₂₋₁₀ alkenyl, as defined for example for the radical R₁ of formula (I), mean that this substituent (radical) is an alkenyl radical having a carbon number of 2 to 10 and having at least one unsaturated carbon-carbon bond. The alkenyl radical can be linear or branched and, if appropriate, cyclic. Alkenyl radicals that have both a cyclic and a linear component are likewise included in this definition. The same applies to other alkenyl radicals such as a C₂₋₄ alkyl radical. If appropriate, alkenyl radicals can also be mono- or polysubstituted with functional groups such as amino, hydroxy, halo, aryl or heteroaryl. Unless otherwise stated, alkenyl radicals preferably do not have any functional groups as substituents. Examples of alkenyl radicals are vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 3-heptenyl, 4-heptenyl, 1-octenyl, 3-octenyl, 5-octenyl, 1-nonenyl, 2-nonenyl.

In the present invention, definitions such as C₂₋₁₀ alkenyl, as defined for example for the radical R₁ of formula (I), mean that this substituent (radical) is an alkenyl radical having a carbon number of 2 to 10 and having at least one unsaturated carbon-carbon bond. The alkenyl radical can be linear or branched and, if appropriate, cyclic. Alkenyl radicals that have both a cyclic and a linear component are likewise included in this definition. The same applies to other alkenyl radicals such as a C₂₋₄ alkenyl radical. If appropriate, alkenyl radicals can also be mono- or polysubstituted with functional groups such as amino, hydroxy, halo, aryl or heteroaryl. Unless otherwise stated, alkenyl radicals preferably do not have any functional groups as substituents. Examples of alkenyl radicals are vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 3-heptenyl, 4-heptenyl, 1-octenyl, 3-octenyl, 5-octenyl, 1-nonenyl, 2-nonenyl.

In the present invention, the definition aryl means that it is an aromatic or heteroaromatic radical. An aromatic radical is an aromatic cyclic hydrocarbon that may consist of one ring or a ring system of several fused rings. The aromatic radical can be, for example, monocyclic, bicyclic or tricyclic. Preferably, the monocyclic aromatic radical forms a 5- or 6-membered ring. Preferably, the bicyclic aromatic ring forms a 9- or 10-membered ring. Preferably, the monocyclic aromatic radical forms a 13- or 14-membered ring. The aryl group preferably contains 3 to 14, more preferably 4 to 6 carbon atoms. If appropriate, aryl radicals can also be mono- or polysubstituted with functional groups such as alkyl, alkenyl, amino, hydroxy, halo, aryl or heteroaryl. Examples of aromatic radicals are phenyl and naphthyl.

In the present invention, the definition heteroaryl means that it is a heteroaromatic radical. Heteroaromatic ring means that in an aromatic radical as defined above, whose ring system is formed by carbon atoms, one or more of these carbon atoms are replaced by heteroatoms such as O, N or S. Examples of heteroaromatic radicals, which are included in the definition of aryl in the present invention, are furanyl, thienyl, oxazolyl, pyrazolyl, pyridyl and indolyl.

In the present invention, the definition of halo, as defined above for the radical R₄ for formula (I), means that it is a chlorine, bromine, iodine or fluorine substituent. Preferably, it is a chlorine or fluorine substituent.

More preferably, the carbonic acid adduct (CAA), (SCAA1), (SCAA2), (SCAA3) or (SCAA4) comprises at least one amine (AM) selected from the group 4-aminobenzoic acid 2-(N,N-diethylamino)ethyl ester (procaine), 4-aminobenzoic acid ethyl ester (benzocaine), 2-(diethylamino)ethyl 4-amino-2-chlorobenzoate (chloroprocaine), 4-amino-3-butoxybenzoic acid 2-diethylaminoethyl ester (oxybuprocaine), (2-(dimethylamino)ethyl)-4-(butylamino)benzoate (tetracaine), N-[3-(4-phenoxymethylphenyl) propyl]morpholine (fomocaine), 2-diethylamino-N-(2,6-dimethylphenyl) acetamide (lidocaine), (RS)—N-(2,6-dimethylphenyl)-1-methylpiperidine-2-carboxamide (mepivacaine), (RS)—N-(2-methylphenyl)-2-(propylamino) propanamide (prilocaine), (RS)-4-methyl-3-[2-(propylamino) propanamido]thiophene-2-carboxylic acid methyl ester (articaine), (+)-1-butyl-N-(2,6-dimethylphenyl)-2-piperidinecarboxamide (bupivacaine), (S)-1-propyl-2′,6′-dimethyl-2-piperidyl-carboxyanilide (ropivacaine), 2-(ethylpropylamino)-2′,6′-butyroxylidide (etidocaine) and 1-(4-butoxyphenyl)-3-piperidin-1-ylpropan-1-one (dyclonine).

Even more preferably, the carbonic acid adduct (CAA), (SCAA1), (SCAA2), (SCAA3) or (SCAA4) comprises at least one amine selected from the group 4-aminobenzoic acid 2-(N,N-diethylamino)ethyl ester (procaine), 2-diethylamino-N-(2,6-dimethylphenyl) acetamide (lidocaine) and (2-(dimethylamino)ethyl)-4-(butylamino)benzoate (tetracaine).

Particularly preferably, the carbonic acid adduct (CAA), (SCAA1), (SCAA2), (SCAA3) or (SCAA4) comprises at least one amine (AM) selected from the group 4-aminobenzoic acid 2-(N,N-diethylamino)ethyl ester (procaine) and 2-diethylamino-N-(2,6-dimethylphenyl) acetamide (lidocaine).

Most preferably, the carbonic acid adduct (CAA), (SCAA1), (SCAA2), (SCAA3) or (SCAA4) comprises 4-aminobenzoic acid 2-(N,N-diethylamino)ethyl ester (procaine) as the amine (AM).

Optionally, the carbonic acid adduct (SCAA1), (SCAA2), (SCAA3) or (SCAA4) comprises at least one salt (S). Preferably, the carbonic acid adduct (SCAA1), (SCAA2), (SCAA3) or (SCAA4) comprises at least one salt (S). Preferably, the carbonic acid adduct (AM) comprises at least one salt (S) when the amine (AM) is procaine or lidocaine.

The at least one salt (S) is preferably a salt which is composed of at least one cation selected from Na⁺, K⁺, Li⁺, Mg²⁺, Zn²⁺, Fe²⁺, Fe³⁺ and Mn²⁺ and at least one anion selected from Cl⁻, Br⁻, I⁻, F⁻, SO₄²⁻, SO₃²⁻, HSO₄⁻, HSO₃⁻, HCO₃⁻, CO₃²⁻, PO₄³⁻, HPO₄²⁻, H₂PO₄⁻, SiO₄⁴⁻, AlO₂⁻, SiO₃⁻ and/or [AlO₂)₁₂(SiO₂)₂]²⁻. More preferably, the cation is selected from Na⁺ and the anion is selected from Cl⁻ or Br⁻. The salt can be formed, for example, by an acid-base reaction of the base (BA) when carrying out step b) with the acid added to the amine (AM), if an acid addition salt of the amine (AM) is employed. The salt (S) can also be added directly in one of steps a), b) and/or c). The direct addition of the salt (S) is preferred if the amine (AM) is not employed in the salt form and/or step b) is not carried out.

The carbonic acid adduct (CAA), (SCAA1), (SCAA2), (SCAA3) or (SCAA4) is producible according to a method comprising, inter alia, steps a), optionally b), c), optionally d), and optionally e).

In step a), a solution (A) is provided which comprises at least one solvent and CO₂ dissolved in the at least one solvent.

The solution (A) comprises at least one solvent and CO₂ in dissolved form. In the context of this invention, CO₂ is understood to mean carbon dioxide. In the context of this invention, CO₂ in dissolved form is understood to mean all forms of CO₂ that it takes on when dissolved. For example, it is known for aqueous solutions that the dissolved CO₂ in the solution can be present in equilibrium as CO₂, as carbonic acid, as singly or doubly deprotonated carbonic acid, i.e. as bicarbonate or carbonate.

The solution (A) is yielded by introducing CO₂ into the at least one solvent. The CO₂ can be introduced into the solvent in all forms known and suitable to the person skilled in the art. Preferably, gaseous or frozen CO₂ in the form of dry ice, more preferably gaseous CO₂, is introduced into the solvent. The CO₂ can also be introduced under pressure, especially when gaseous CO₂ is introduced into the solution. In this context, introduction under pressure means that a pressure greater than atmospheric pressure, preferably greater than 1.01325 bar, is used. For this purpose, the CO₂ can be introduced into the solvent in a container which isolates the solvent from the environment in such a way that a pressure can be generated in the container, in particular by supplying the CO₂, which pressure is above atmospheric pressure, preferably above 1.01325 bar. Introducing CO₂ into the solution, especially in gaseous form, can be done in one step or at intervals.

The person skilled in the art may utilize any suitable solvent in step a). Preferably, a polar protic solvent is employed as the solvent, more preferably the solvent is water. The solvent can be employed in different degrees of purity depending on the intended use of the carbonic acid adduct (CAA), (SCAA1), (SCAA2), (SCAA3) or (SCAA4). For example, water with the purity grade “Water for Injection (aqua ad iniectabilia)” can be used if the carbonic acid adduct (SCAA1), (SCAA2), (SCAA3) or (SCAA4) is to be employed for pharmaceutical-medicinal purposes.

Step a) may comprise substep a1), wherein the solvent is cooled to 3 to 8° C., preferably to 5° C., preferably before the introduction of the CO₂. Cooling can be carried out by means of any method known to the person skilled in the art, and identified as suitable. For example, cooling can be achieved by storing the solvent in a refrigerator for a sufficiently long time until the solvent reaches the target temperature. External cooling can also be used, for example.

Step a) may comprise substep a2), wherein the CO₂ is introduced into the solvent, preferably until a saturation concentration of 3 to 10 g/l is reached, more preferably until a saturation concentration of 4.5 to 7.5 g/l is reached, based on the total volume of the solution. Preferably, the pH of the solution after saturation with CO₂ is ≤3.0 to ≤6.0, even more preferably ≤4.3 to ≤4.8. Preferably, the CO₂ is dissolved in substep a2) under pressure, wherein the pressure is 1.5 to 10 bar, more preferably 1.9 to 7 bar, even more preferably 2 to 5 bar.

Step a) may comprise substep a3), wherein the solution (A) preferably yielded in substep a2) is stored at 1 to 10° C., preferably for at least 30 min, more preferably for at least 50 min, even more preferably for at least 60 min; up to a maximum of 5 d (120 h). Preferably, solution (A) preferably yielded in substep a2) is stored at 3 to 8° C. for at least 30 min, more preferably for at least 50 min, even more preferably for at least 60 min; up to a maximum of 5 days (120 h).

Preferably, step a) comprises all substeps a1), a2) and a3).

Preferably, the substeps a1), a2) and a3) are carried out in the order a2) follows a1) and a3) follows a2).

Optionally, step b) can be carried out, wherein the base (BA) other than the amine (AM) is dissolved in solution (A) to yield solution (A1). Preferably, base (BA) is a bicarbonate or a carbonate, more preferably a bicarbonate, even more preferably sodium bicarbonate.

In step c), the at least one amine (AM) is dissolved in the solution (A) or (A1) to yield the solution (B).

The at least one amine (AM), as defined above, can be employed in step c) both in neutral form and in the form of a salt. Optionally, the at least one amine (AM) can also be used as a mixture of the neutral form of the amine (AM) with the salt form of the amine (AM). Therefore, the at least one amine (AM) may comprise the neutral amine (AM) and/or the salt form of the at least one amine (AM). Preferably, the salt form of the at least one amine (AM) is an acid addition salt, preferably the acid addition salt is a hydrochloride, hydrobromide, hydroiodide, bisulfate, bisulfite, biphosphate, hydromesylate, hydrotosylate, hydroacetate, hydroformate, hydropropanoate, hydromalonate, hydrosuccinate, hydrofumarate, hydroxalate, hydrotartrate, hydrocitrate, hydromaleate, more preferably a hydrochloride or hydrobromide, even more preferably a hydrochloride of the at least one amine (AM).

Preferably, the concentration of the amine (AM) in the solution (B) is 0.01 to 0.25 g/ml, preferably 0.03 to 0.20 g/ml, more preferably 0.08 to 0.15 g/ml.

Step c) may comprise substep c2), wherein the at least one amine (AM) is dissolved in the solution (A), or when carrying out step b), in the solution (A1) to yield the solution (B).

In one possible embodiment, the ratio of the amine (AM) to the base (BA), when carrying out step b), in the solution (B) is 2:1 to 5:1, more preferably 3:1 to 4:1, even more preferably 3.23:1 to 3.26:1 [g/g]].

In a further possible embodiment, the molar ratio of the amine (AM) to the base equivalents of the base (BA), when carrying out step b), in the solution (B) is 0.8:1 to 1.5:1, preferably 1.2:1, more preferably 1:1. Base equivalents in this context means that when using a monovalent base, such as NaHCO₃, the molar ratio of the base (BA) to the amine (AM) corresponds to the ratio given above. When using a divalent base (BA), such as Na₂CO₃, only half the amount of base is required to introduce the same amount of base equivalents, based on the amount of moles of the base (BA) compared to using a monovalent base. For example, at a ratio of 1:1, using 10 mmol of the amine (AM), 10 mmol of NaHCO₃ are required, but only 5 mmol of Na₂CO₃.

In a further embodiment, step b) is carried out and in substep c1) the amine (AM) is added in the form of the acid addition salt, wherein amine (AM) with the acid bound to it is added in such an amount that the acid bound to the amine (AM) is able to neutralize the base (BA) to such an extent that the solution (B) assumes a pH of 6 to 8.

Step c) may comprise substep c2), wherein the solution (A) is added to the solution (B) to yield the solution (B1).

Preferably, the concentration of the amine (AM) in the solution (B1) is 0.01 to 0.25 g/ml, preferably 0.03 to 0.20 g/ml, more preferably 0.08 to 0.15 g/ml.

Step c) may comprise substep c3), wherein the solution (B) or, when carrying out substep c2), the solution (B1) is enriched with CO₂. Preferably, the solution (B) is enriched with 2.5 g/l to 9 g/l, more preferably with 5 to 7.5 g/l of CO₂.

Step c) may comprise substep c4), wherein the solution (B) or, when carrying out substep c2), the solution (B1) is stored at 1 to 10° C., preferably 3 to 8° C., for at least 1 h, preferably 24 h to 120 h, even more preferably 24 to 72 h.

Step c) may comprise substep c5), wherein the solution (B) or, when carrying out substep b2), the solution (B1) is enriched with CO₂ to a total concentration of at least 6 g/l, preferably at least 10 g/l, more preferably at least 12 g/l, even more preferably at least 14 g/l and very particularly preferably at least 15 g/l. Preferably, in substep c5), a further 0.4 to 4.7 g/l, more preferably 1 to 3.5 g/l of CO₂ are introduced or dissolved into the solution (B) or (B1) until the required total concentration is reached.

The term “total concentration” refers here to the total concentration of dissolved CO₂ in the solution (B) or (B1), including the CO₂ bound in the carbonic acid adduct (CAA). The total concentration results additively from the weight increase of the solution due to the added CO₂ in all previous enrichment steps a2) and/or c3), as far as stated, and c5), without taking into account CO₂, which is optionally added to the solution in the form of bicarbonate or carbonate as the base (BA).

Enriching the solution (B) or the solution (B1) in substep c5) with CO₂ to the required total concentration can be carried out at a pressure of 2.5 to 10 bar, preferably 4 to 10 bar, more preferably 5 to 10 bar, even more preferably 6 to 10 bar, most preferably 6.5 to 10 bar. Preferably, the solution (B) or (B1) has a temperature of 3 to 8° C., more preferably 5° C., when enriched with CO₂ in substep c5).

The enrichment of the solution (B) or (B1) in substeps c3) and c5) can be carried out in the same manner as described for step a).

Preferably, the pH of the solution (B) or, when carrying out substep c2), of the solution (B1) after carrying out step c5) is ≤7.0.

Preferably, step c) comprises all substeps c1), c2), c3), c4) and c5).

Preferably, substeps c1), c2), c3), c4) and c5) are carried out in the order c2) follows c1), c3) follows c2), c4) follows c3), c5) follows c4).

The production of the stabilized carbonic acid adducts (SCAA1) and (SCAA4) comprises step d). In step d), the solution yielded after completion of step c) is frozen. Preferably, in step d), the solution B) or, after carrying out substep c2), the solution (B1) is frozen.

The solution, preferably the solution (B) or (B1), which is subjected to step d) has a CO₂ content of at least 6 g/l, preferably at least 7 g/l, more preferably at least 8 g/l, particularly preferably 8.1 g/l to 9.5 g/l in the production of (SCAA1).

Preferably, the solution yielded after completion of step c), preferably the solution (B) or (B1), is frozen in step d) at −100° C. to −20° C., more preferably at −90° C. to −30° C., even more preferably at −80 to −40° C., and most preferably at −70 to −50° C. in step d).

The freezing of the solution yielded after completion of step c), preferably the solution (B) or (B1), in step d), can in principle be carried out by all methods known to the person skilled in the art and identified as suitable. For example, freezing can be carried out by transferring the solution yielded in step c) into a suitable vessel immersed in a cooling medium. Preferably, the vessel has a flask shape. Preferably, the vessel containing the solution yielded in step c) is immersed in the cooling medium at an angle of 40°. The cooling medium can, for example, consist of a solvent such as methanol, ethanol or acetone, which is brought to the desired temperature by adding dry ice or by suitable cooling equipment such as cryostats.

Preferably, the freezing in step d) takes place at atmospheric pressure, more preferably at 1.01325 bar.

Preferably, the solution yielded after completion of step c), preferably the solution (B) or (B1), in step d), is frozen within 0.3 to 60 minutes, more preferably within 1 to 30 minutes, even more preferably within 1.1 to 10 minutes, particularly preferably within 1.5 to 5 minutes.

The solution yielded after completion of step c), preferably the solution (B) or (B1), is frozen in step d) preferably at a cooling rate of 10 to 100 K/min, more preferably at 20 to 80 K/min, even more preferably at 30 to 70 K/min, and particularly preferably at 40 to 60 K/min.

Preferably, the vessel in which the solution yielded after completion of step c), preferably the solution (B) or (B1), is located in step d) during the freezing process, is rotated in the cooling medium at 10 to 1000 rpm, more preferably at 50 to 600 rpm, even more preferably at 100 to 400 rpm and particularly preferably at 200 to 300 rpm.

Freezing in step d) can be carried out using the shell freeze method.

The production of the stabilized carbonic acid adducts (SCAA1) and (SCAA4) comprises step e). In step e), the solution frozen in step d), preferably the solution (B) or (B1), is stored at −100 to 0° C. for no longer than 4 days.

Preferably, the solution frozen in step d), preferably the solution (B) or (B1), is stored in step e) for 1.5 to 4 days, more preferably for 2.5 to 4 days.

Preferably, the solution frozen in step d), preferably the solution (B) or (B1), is stored in step e) at −50 to 0° C., more preferably at −30 to −5° C., even more preferably at −25 to ~10° C., particularly preferably at −20 to ~15° C.

Storing in step e) can in principle take place at the defined temperature in any cooling device known to the person skilled in the art. For example, storing can be carried out in a freezer or a freezer room.

The method by which the carbonic acid adduct is producible comprises step f). The method by which the carbonic acid adduct (SCAA1) or (SCAA3) is producible may comprise a further step f). Step f) is carried out after step e) or j). Here, in step f), the solution stored in step e) or j), preferably the solution (B) or (B1), is dried to yield the dried carbonic acid adduct (CAA), (SCAA1) or (SCAA3).

Preferably, in step f), the water is removed from the solution stored in step e) or j), preferably the solution (B) or (B1), to a residual content of <0.8 wt. %, more preferably to a residual content of <0.1 wt. %, based on the total weight of the dried carbonic acid adduct (CAA).

Preferably, in step f), CO₂ not bound in the carbonic acid adduct (SCAA1) or (SCAA3) is removed from the solution stored in step e) or j), preferably (B) or (B1), to a residual content of <0.8 wt. %, more preferably to a residual content of <0.1 wt. %, based on the total weight of the dried carbonic acid adduct (SCAA1) or (SCAA3).

Drying can be carried out using any method known to the person skilled in the art, and identified as suitable. Drying is preferably carried out by freeze-drying, also called lyophilization. In the case of the freeze-drying method, step d) represents the freezing step and steps e) or j) represent the maturation step. Lyophilization can also be performed as plate lyophilization or spray freeze-drying.

The pressure during drying in step f) is preferably 0.01 to 30 mbar, preferably 0.02 to 20 mbar, more preferably 0.03 to 10 mbar, even more preferably 0.03 to 0.5 mbar, and most preferably 0.05 to 0.1 mbar. The pressure is preferably maintained throughout the entire drying process. Preferably, the pressure defined above is reached during drying within 7 h, more preferably within 5 h, and particularly preferably within 4 h from the start of evacuation.

The person skilled in the art can determine the end point of drying from the temperature profile recordings. Preferably, the total drying time in step f) is 10 to 60 h, more preferably 30 to 55 h, particularly preferably 41 to 52 h. The total drying time is defined as the time between the completion of storage in step e) or j) and the completion of drying in step f).

Preferably, the temperature during the entire drying in step f) is 0 to 20° C., preferably 4 to 18° C., more preferably 8 to 16° C.

The method for producing the carbonic acid adduct (SCAA1) comprises step g1), adding polyvinylpyrrolidone to the carbonic acid adduct (CAA) yielded in the preceding steps after carrying out step e) to yield the stabilized carbonic acid adduct (SCAA1).

In step g1), the weight ratio of polyvinylpyrrolidone (PVP) to the carbonic acid adduct (CAA) is preferably 10:1, more preferably 5:1, particularly preferably 3:1.

The addition of polyvinylpyrrolidone in step g1) can be carried out either with polyvinylpyrrolidone as a solid or as a solution in water. The concentration of polyvinylpyrrolidone in the added solution is adjusted according to the required amount of polyvinylpyrrolidone in step g1), depending on the weight ratio of polyvinylpyrrolidone to the carbonic acid adduct (CAA). Preferably, the concentration of polyvinylpyrrolidone in water is 0.5 to 20 wt %, more preferably 0.9 to 18 wt %. Preferably, the concentration of the carbonic acid adduct (CAA) is 0.15 wt % to 5.7 wt %.

In one embodiment, preferably for inhalation, the weight ratio of polyvinylpyrrolidone (PVP) to carbonic acid adduct (CAA) is preferably 3:1, and the concentration of polyvinylpyrrolidone (PVP) is 0.9 wt % in water. Preferably, the concentration of the carbonic acid adduct (CAA) is 0.15 wt %.

In one embodiment, preferably for buccal application, the weight ratio of polyvinylpyrrolidone (PVP) to the carbonic acid adduct (CAA) is preferably 3:1, and the concentration of polyvinylpyrrolidone (PVP) is 17.1 wt % in water. Preferably, the concentration of the carbonic acid adduct (CAA) is 5.7 wt %.

In one embodiment, preferably for parenteral application, the weight ratio of polyvinylpyrrolidone (PVP) to the carbonic acid adduct (CAA) is preferably 3:1, and the concentration of polyvinylpyrrolidone (PVP) is 6 wt %. Preferably, the concentration of the carbonic acid adduct (CAA) is 2 wt %.

Preferably, step g1) is carried out after step f) following step e). In this embodiment, the carbonic acid adduct (CAA) and the polyvinylpyrrolidone are mixed in the form of their solids, preferably as a powder, to yield the stabilized carbonic acid adduct (SCAA1).

In step g1), the polyvinylpyrrolidone (PVP) preferably has an average molecular weight of 10,000 to 40,000 g/mol, more preferably of 15,000 to 35,000 g/mol, even more preferably of 24,000 to 33,000 g/mol, particularly preferably of 17,000 g/mol to 32,000 g/mol.

The method for producing the carbonic acid adduct (SCAA2) comprises step g2), adding polyvinylpyrrolidone to the solution (B) yielded in step c) to yield the stabilized carbonic acid adduct (SCAA2),

In step g2), the weight ratio of polyvinylpyrrolidone (PVP) to the carbonic acid adduct (CAA) is preferably 1:1 to 20:1, more preferably 4:1 to 10:1, particularly preferably 5:1.

The addition of polyvinylpyrrolidone in step g2) can be carried out either with polyvinylpyrrolidone as a solid or as a solution in water. The concentration of polyvinylpyrrolidone in the added solution is adjusted according to the required amount of polyvinylpyrrolidone in step g2), depending on the weight ratio of polyvinylpyrrolidone to the carbonic acid adduct (CAA). Preferably, the concentration of the carbonic acid adduct (CAA) is 0.15 wt % to 5.7 wt % in water. Preferably, the concentration of polyvinylpyrrolidone in water is 0.75 to 30 wt %, more preferably 1.5 to 28.5 wt %.

When producing the stabilized carbonic acid adduct (SCAA2), the content of CO₂ in the solution (B) subjected to step c) is at least 10 g/l, preferably at least 12 g/l, more preferably at least 14 g/l, and even more preferably at least 14 to 15 g/l, and the amine (AM) can also be employed in the form of a salt.

In step g2), the polyvinylpyrrolidone (PVP) preferably has an average molecular weight of 15,000 to 35,000 g/mol, more preferably of 24,000 to 33,000 g/mol, and particularly preferably of 16,000 g/mol to 27,000 g/mol.

The method for producing the carbonic acid adduct (SCAA3) comprises step g3), adding polyvinylpyrrolidone to the solution (B) yielded in step c) to yield the solution (C).

Preferably, in step g3), the weight ratio of polyvinylpyrrolidone (PVP) to carbonic acid adduct (CAA) is 6:1 to 1:0.5, more preferably 3:1, particularly preferably 1.5:1.

Preferably, polyvinylpyrrolidone is added as a solution. More preferably, polyvinylpyrrolidone is added in water as a solvent. Preferably, in this solution, the weight ratio (w/w) of polyvinylpyrrolidone:water is 1.1 to 0.75, more preferably 0.9 to 0.8, particularly preferably 0.85.

Preferably, in step g3) one or more further additives are added, selected from a monosaccharide, more preferably galactose and glucose.

The method for producing the carbonic acid adduct (SCAA3) comprises step h), freezing the solution yielded after completion of step g3).

The CO₂ content in the solution subjected to step h) is at least 6 g/l, preferably at least 10 g/l, more preferably at least 12 g/l, even more preferably at least 14 g/l, and most preferably at least 15 g/l. The solution subjected to step h) is, if necessary, enriched with further CO₂ until the concentrations specified above are reached.

The method for producing the stabilized carbonic acid adduct (SCAA3) comprises step j), storing the solution frozen in step h) at −100 to 0° C. for no longer than 4 days.

The method for producing the stabilized carbonic acid adduct (SCAA4) comprises step k), drying a CO₂-saturated solution comprising the carbonic acid adduct (CAA) from step f), and polyvinylpyrrolidone.

Wherein the CO₂-saturated solution which is dried in step k) has a CO₂ concentration of at least 1.2 g/l of CO₂, more preferably of at least 1.5 g/l of CO₂.

In one embodiment, step k) comprises the substeps of
k1) dissolving polyvinylpyrrolidone in water to yield the solution (Sk1);
optionally, k2) enriching the solution (Sk1) with CO₂; preferably, enriching the solution (Sk1) with CO₂ in step k2) takes place under atmospheric pressure.
k3) dissolving the carbonic acid adduct (CAA) in the solution (Sk1) yielded in step k2) or k1) to yield the solution (Sk3); preferably, the mass ratio of the carbonic acid adduct (CAA) to polyvinylpyrrolidone is 1:1.5.
k4) saturating the solution (Sk3) with CO₂. Wherein the saturation is preferably carried out under overpressure, up to a concentration of at least 1.2 g/l of CO₂.

In a further embodiment, step k) comprises the substeps of
k1) dissolving polyvinylpyrrolidone in water to yield the solution (Sk1);
k5) dissolving the carbonic acid adduct (CAA) in water saturated with CO₂ to yield the solution (Sk4);

The concentration of polyvinylpyrrolidone in water in step k1) in the solution (Sk1) is ≤850 g/l.

The method described above for producing the stabilized carbonic acid adducts (SCAA1), (SCAA2), (SCAA3) and (SCAA4) are a further aspect of the invention.

(SCAA2) is always present as a solution.

(SCAA3) and (SCAA4) are structurally identical and show the same infrared (IR) spectra, insofar as they are comparable embodiments, e.g., with the same bases. The only difference here is the manufacturing methods.

### Formulations

The invention also comprises pharmaceutical preparations, which is, in the context of this invention, understood to mean a composition which comprises the stabilized the stabilized carbonic acid adduct (SCAA1), (SCAA2), (SCAA3) or (SCAA4) and, in addition, may comprise further excipients or additives which are suitable for pharmaceutical-medical use.

In addition, the pharmaceutical preparations according to the invention may comprise further bases other than the amine (AM) and may be different from the base (BA). The person skilled in the art can generally select the additives depending on the intended purpose. The desired form of application will be taken into account.

The pharmaceutical preparations can in principle be present in any suitable dosage form. For example, the pharmaceutical preparations can be in the form of capsules, tablets, coated tablets, lyophilizates, globuli velati, solutions, dipping solutions for wound dressings, ointments, creams, gels, pastes, compresses or plasters containing active ingredients.

The pharmaceutical preparations can in principle be administered in any suitable form of application. The person skilled in the art will select a suitable dosage form according to the intended form of application. For example, the pharmaceutical preparations can be administered parenterally, lingually, sublingually, buccally, orally, by inhalation, by injection, as a patch, cutaneously, comprising at least dermal application, ocular application, nasal application, rectal application and vaginal application. The pharmaceutical preparations are preferably administered buccally, by inhalation, parenterally or orally.

When preparing the pharmaceutical preparations, the person skilled in the art can, in principle, use the methods known in the prior art.

Preferably, the temperature of the mixture of the carbonic acid adduct (SCAA1), (SCAA2), (SCAA3) or (SCAA3) and the excipients used and, if appropriate, further bases during the production of the pharmaceutical preparations (PP) is less than 60° C., preferably less than 50° C., more preferably 0 to 50° C.

When preparing the pharmaceutical preparations, optionally in ointment form, dispersion can also be employed, preferably using an ointment mixer. A speed of <2000 rpm is preferably used.

The pharmaceutical preparations may contain one or more preservatives. Preferably selected from the group consisting of methyl hydroxybenzoate, propyl hydroxybenzoate, potassium sorbate, sodium sorbate, sorbic acid, 4-hydroxybenzoic acid methyl ester, 4-hydroxybenzoic acid propyl ester,

The stabilized carbonic acid adducts (SCAA1), (SCAA3) and (SCAA4), preferably (SCAA3), can be formulated in one embodiment as an ointment with an aqueous base. Ointments with an aqueous base can generally be produced according to generally known principles.

The ointments with an aqueous base preferably comprise at least one of the excipients selected from the group consisting of hydroxyethylcellulose gel, hydroxyethylcellulose mucilage, sorbitan monooleate, glycerol monooleate, (C₁₀-C₂₅) alkylOH, ceteareth-80, glyceryl stearate, cetearyl alcohol and sodium cetylstearyl sulfate.

The carbonic acid adduct (SCAA1), (SCAA3) or (SCAA4) can be triturated to form a powder before processing into the oral dosage forms described below, such as tablets or capsules or semi-solid dosage forms, alone or in the presence of other excipients or bases. In principle, the person skilled in the art can use the technical means that are suitable and known for the respective purpose. For example, mortars, planetary mills or similar suitable devices can be employed for trituration steps. Preferably, a technical aid is employed in the trituration steps that keeps the mechanical stress on the stabilized carbonic acid adducts as low as possible. Triturating is preferably done with a mortar. Preferably, the carbonic acid adducts (SCAA3) and (SCAA4) are subjected to trituration using a planetary mill.

The powder thus yielded can then be pressed into tablets, filled into commercially available capsules, or mixed with suitable excipients and processed to form semi-solid dosage forms.

In one embodiment, the stabilized carbonic acid adduct (SCAA3), preferably after step f), can be formulated or compressed directly into tablet form, preferably without the need for further excipients to support the formulation of the tablet form. Preferably, in this embodiment, polyvinylpyrrolidone (PVP) having an average molecular weight of 24,000 to 33,000 g/mol, more preferably of 17,000 g/mol to 30,000 g/mol, is used in step g3).

One embodiment of the pharmaceutical production relates to a pharmaceutical preparation comprising the carbonic acid adduct (SCAA1), (SCAA3), (SCAA4) and is administered orally. In this embodiment, the pharmaceutical preparation is preferably administered in capsules, more preferably in hard gelatin or cellulose capsules, particularly preferably in hard gelatin capsules. Likewise, the pharmaceutical preparation in this embodiment can be administered in tablet form.

Preferably, the pharmaceutical preparation in this embodiment comprises at least one excipient (H), preferably selected from starch, in particular corn starch and/or rice starch, dextran, cellulose esters and SiO₂.

The stabilized carbonic acid adducts (SCAA1) and (SCAA2) as yielded in steps g1) and g2) in the form of a solution can, in one embodiment, be used directly as a solution, preferably for oral application or as a spray. In this stabilized form as (SCAA1) and as (SCAA2), the carbonic acid adduct (CAA) can be repeatedly dispensed from the corresponding storage vessel, so that a multi-dispensing system can be implemented.

The stabilized carbonic acid adduct (SCAA2) yielded as in step g2) in the form of a solution can, in one embodiment, be used directly as a solution, preferably for inhalational application, as a drinking solution, or as a parenteral.

The methods described above for preparing the stabilized carbonic acid adducts (SCAA1), (SCAA2), (SCAA3) and (SCAA4) are a further aspect of the invention.

Furthermore, the invention comprises a kit.

The kit comprises:
a) carbonic acid adduct (CAA) produced as defined for (SCAA1) or disclosed in WO2019048590 (A1).
b) polyvinylpyrrolidone;
optionally, c): an isotonic solution or non-isotonic solution, preferably an isotonic solution selected from 0.9% (w/w) NaCl solution, or a full electrolyte solution, preferably selected from Stereofundin, Stereofundin ISO, Ringer's solution, Ringer's acetate, Ringer's lactate, Deltajonin, Jonosteril, and Jonosteril malate,
wherein carbonic acid adduct (CAA) and the polyvinylpyrrolidone are optionally present as a solid mixture.

The proportions in the solid mixture are as for (SCAA1).

Preferably, a full electrolyte solution is an isotonic solution that is similar in composition to the electrolyte profile of human blood plasma and has the same osmolarity as blood plasma. A full electrolyte solution preferably comprises at least one cation and one anion, more preferably all of the following ions: Na⁺, K⁺, Cl⁻, Ca²⁺, Mg²⁺, acetate, malate, lactate. The concentration of ions in the full electrolyte solution is adjusted so that the full electrolyte solution is isotonic, as listed above.

### Medical Applications

The stabilized carbonic acid adducts (SCAA1), (SCAA2), (SCAA3) and (SCAA4) or the above-mentioned formulations are applicable for use in medicine.

Furthermore, the stabilized carbonic acid adduct (SCAA1), (SCAA2), (SCAA3) and (SCAA4) are applicable for use in a method of anesthesia, of analgesia, of concomitant treatment of cancer, of treatment of amyotrophic lateral sclerosis (ALS), of anti-inflammatory treatment, of supporting wound healing, in particular in burns, open wounds and scars, of treatment of neurogenic inflammations such as multiple sclerosis, MMN (multifocal motor neuropathy), of treatment of sinusitis, of treatment of asthma, of treatment of rheumatoid arthritis, of treatment of Alzheimer's disease, of treatment of dementia, of supporting convalescence and of supporting anti-aging, of treatment of burnout syndrome, of treatment of osteoarthritis, of treatment of polyarthritis, of treatment of pain syndrome and general pain, of pre- and post-operative treatment (also for bone fractures), of preventive and rehabilitation medicine, of treatment of zoster neuralgia, of accompanying treatment of zoster neuralgia, of treatment of diseases of the abdominal organs such as liver, gallbladder, pancreas, intestines, of treatment of gastrointestinal diseases (ulcerative colitis, Crohn's disease), of treatment of ankylosing spondylitis, of treatment of chronic musculoskeletal pain, of treatment of diabetes (improvement of blood sugar levels), of reducing edema, fatigue, long-COVID, of improving microcirculation, and of comedication to opioids.

### EXAMPLES

### 1. Exemplary Embodiment 1, Example of the Production of an Embodiment of the Stabilized Carbonic Acid Adduct (SCAA3)

### 1.1 Materials:

Amine (AM): 64.99 g of procaine hydrochloride (e.g., ultra pure, for use as a pharmaceutical active ingredient; Ph. Eur. or in a quality suitable for this purpose)
Base (BA): 20.01 g of sodium bicarbonate (e.g., ultra pure, or in a quality suitable for this purpose),
PVP: 127.5 g of PVP (PVP-polyvinylpyrrolidone, also called polyvidone or povidone)
Solvent: 530 to 850 ml of water (Water for Injection)
CO₂: at least 7.5 g/l of carbon dioxide from pressurized steel cylinders (CO₂ of suitable quality).
Dry ice for the preparation of cooling mixtures and for cooling
Methanol, technical for the production of cooling mixtures

### 1.2 Step a)

Water (e.g., Water for Injection) is filled into a cleaned plastic pressure bottle up to the mark (approx. 800 to 900 ml) and pre-cooled for at least 1 h in the refrigerator (3 to 8° C.) or by means of external cooling to 5° C.

A carbonic acid solution saturated with carbon dioxide is prepared. For this purpose, CO₂ is introduced into the pre-cooled water at intervals under pressure (1.6 to 8 bar). The hissing (escaping gas through the pressure relief valve) indicates saturation of the solution with CO₂. The saturation is controlled by weight until 4.0 to 6.0 g of CO₂ (corresponding to 4.5 to 7.5 g/l) are dissolved. The saturated solution has a pH of ≤4.3 to 4.8. This carbonated water is sealed immediately and stored in the refrigerator for at least 1 h.

### 1.3 Step b)

In a second plastic pressure bottle, 20.01 g of sodium bicarbonate is placed, mixed with 265 ml of cooled CO₂-containing water and dissolved while swirling.

### 1.4 Step c)

To this solution, the equivalent amount of solid procaine hydrochloride is added at constant temperature, forming a nearly neutral solution which, after adding a further 265 ml of cold carbonated water, yields a clear, weakly acidic solution. The solution is enriched with CO₂. The solution prepared in this way is stored in the refrigerator for at least 1 h.

### 1.5 Step gx)

The PVP solution is prepared in a third plastic pressure bottle fitted with a magnetic stirrer. For this purpose, 150 ml of water (aqua ad iniectabilia) is added into the bottle and 127.5 g of PVP is added in portions. The stirring rate is adjusted as needed. The full dissolution is verified. The solution must be clear.

### 1.6 Step g3)

To prepare the final reaction solution, the ProcCluster solution is added to the third plastic pressure bottle containing the PVP solution, thus combining both solutions.

The solution is then conditioned again with CO₂ until a CO₂ concentration of at least 7 g/l is reached in the solution. The solution is then stored in the refrigerator for at least 1 h and up to a maximum of 5 days.

### 1.5 Step d)

Round-bottomed flasks are pre-cooled. For freezing, the reaction solution is measured in a pre-cooled measuring cylinder, transferred in portions into round-bottomed flasks and frozen by immersion in a dry ice/methanol cooling mixture (<−60° C.) according to the shell-freezing method within 1.5-3.5 min per flask (~200 rpm). The immersion angle of the flask on the rotary evaporator is set to approx. 40°.

### 1.6 Step e)

The flasks containing the frozen material are closed with a ground-glass stopper and temporarily stored in a freezer at −15 to −20° C. for 2 to 4 days.

### 1.7 Step f)

The thus temperature-controlled flasks are encased in pre-cooled styrofoam containers and immediately connected individually to an evacuated (0.060±0.01 mbar, approx. −46° C., leak test) freeze-dryer via a flexible rubber cone. The valves are carefully opened and the individual flasks are placed under vacuum. Finally, all flasks must be evacuated.

To monitor the process, temperature sensors are placed at the bottom of the styrofoam casing, which record the entire temperature profile during the entire drying process. Before starting lyophilization, the temperature sensors indicate temperatures of <−5° C.

During lyophilization the pressure is 0.07±0.02 mbar. This sublimation pressure is reached within 4 h and maintained throughout the lyophilization time. The cooling chamber is kept at a temperature of 9 to 15° C. throughout the drying process. The end point of lyophilization is determined graphically from the temperature profile recordings. The overall drying time did not exceed 52 h.

After the evaporation flasks have adjusted to room temperature, the substance is homogenized twice in the Pulverisette for 10 s each at 2000 rpm.

The dry lyophilizate is transferred into a brown glass container with a twist-off lid and stored in the refrigerator at 0 to 15° C.

### 2. Examples of Pharmaceutical Preparations (PP)

### 2.1 Capsules and Tablets

The composition of the pharmaceutical preparation (PP) according to the invention in the embodiment as a capsule or tablet for procaine as the amine (AM) is described below by way of example. For the production of the capsules, commercially available snap-fit capsules in the commercially available sizes (5 to 000) can be used, which are filled with the powder containing the stabilized carbonic acid adduct (SCAA1, SCAA3, SCAA4), comprising procaine as the amine (AM) (trituration of the active ingredient stabilized carbonic acid adduct (SCAA), comprising procaine as the amine (AM), if appropriate, with additives, fillers, and glidants).

Direct tableting was carried out with the homogenized product without additives. For example, the tablets produced with 190 mg of active ingredient showed no changes even after 4 months of storage in the refrigerator and at room temperature and are therefore stable (FIG. 8). The stability was investigated using IR spectroscopy. No procaine was detected by IR spectroscopy within the 4-month period. The content was determined at room temperature using UV/VIS spectroscopy. For prescription drugs, a tolerance range of ±15% is prescribed according to European Pharmacopoeia 2.9.6 based on the total content including by-products.

### 2.2 Ointments

The composition of the pharmaceutical preparation (PP) according to the invention in the embodiment as an ointment is explained below, using procaine as the amine (AM) in the stabilized carbonic acid adduct (SCAA1, SCAA3, SCAA4) as an example. When preparing the ointment, the applicable and generally accepted pharmaceutical rules for the production of (compounded) drugs are applied (for example, European Pharmacopoeia, German Pharmaceutical Codex). Greater shear forces are avoided in the production of the ointment. In addition, the temperature during preparation is also kept locally below 60° C. The mortared stabilized carbonic acid adduct (SCAA1) or stabilized carbonic acid adduct (SCAA3, SCAA4) comprising procaine as the amine (AM) is introduced into the ointment base, for example Vaseline, in a mortar or an evaporating dish, which are temperature-controlled by means of a water bath heated to 40 to 45° C. Alternatively, it is also possible to use electrical mixing systems, such as those used in normal pharmacy operations.

Base cream, wool wax or hydrogels can also be used as the ointment base. Temperature control during preparation is not necessary.

When using SCAA3 or SCAA4, prior triturating is not necessary.

### 2.3 Buccal, Lingual, Sublingual Application

The composition of the pharmaceutical preparation (PP) according to the invention in the embodiment as buccal, lingual, sublingual application is explained below, using procaine as the amine (AM) in the stabilized carbonic acid adduct (SCAA1, SCAA3, SCAA4) as an example. When preparing the applications, the applicable and generally accepted pharmaceutical rules for the production of (compounded) drugs are applied (for example, European Pharmacopoeia, German Pharmaceutical Codex).
SCAA1: carbonic acid adduct (CAA)+PVP=1: 3/1:5→mix→in spray bottle→dissolve directly before first use
Dissolve 285 mg of (carbonic acid adduct (CAA)) and 852 mg of PVP25 (mass ratio 1:3) in 5 ml CO₂ water (e.g., commercially available mineral water, containing CO₂) and administer by means of a pump spray bottle. PVP=polyvinylpyrrolidone, also called polyvidone or povidone.
Other PVP products can be employed, for example PVP30, PVP25 and PVP17→other ratios (useful from 1:1 to 1:10, based on the amount of ProcCluster, applies to all PVP used).

The production of the carbonic acid adduct (CAA) is disclosed in WO2019048590 (A1).

SCAA3/SCAA4→as a spray in a spray bottle→dissolve directly before first use

Dissolve 710 mg of stabilized carbonic acid adduct (SCAA3) or (SCAA4) in 5 ml of CO₂ water (e.g., commercially available mineral water containing CO₂) and administer by means of a pump spray bottle.

This amount corresponds to a duration of use of about one week. The solution is stable for a period of 9 days after production.

### Globuli Velati

Production according to Pharm. Eur.; (Not for homeopathic use, contains active ingredient.)

### Lingual Tablets

See tableting above with correspondingly lower amount of active ingredient

### 2.4 Application by Inhalation

The composition of the pharmaceutical preparation (PP) according to the invention in the embodiment as an application by inhalation is explained below, using procaine as the amine (AM) in the stabilized carbonic acid adduct (SCAA1, SCAA3, SCAA4) as an example. When preparing the applications, the applicable and generally accepted pharmaceutical rules for the production of (compounded) drugs are applied (for example, European Pharmacopoeia, German Pharmaceutical Codex).

SCAA1: carbonic acid adduct (CAA)+PVP=1:3→mix→e.g. in crimp top bottle→dissolve directly before first use

25 mg of (carbonic acid adduct (CAA)) and 75 mg of PVP25 (mass ratio 1:3) dissolved in 5 ml 0.9% sodium chloride solution. PVP=polyvinylpyrrolidone, also called polyvidone or povidone The solution is placed in a nebulizer, e.g., pariboy, and administered as described in the device's instructions for use, e.g., via a mask or mouthpiece.

Other PVP products can be employed, for example PVP30, PVP25 and PVP17→other ratios (useful from 1:1 to 1:10, based on the amount of ProcCluster, applies to all PVP used).

SCAA3/SCAA4→in crimp top bottle→directly before use

Dissolve 37.5 mg of stabilized carbonic acid adduct in 5 ml of water and place in a nebulizer, e.g., pariboy, and administer as described in the device's instructions for use, e.g., via mask or mouthpiece.

The solution is stable for a period of 24 h after production.

### 2.5 Parenteral Administration

The composition of the pharmaceutical preparation (PP) according to the invention in the embodiment as a parenteral application is explained below, using procaine as the amine (AM) in the stabilized carbonic acid adduct (SCAA1, SCAA2, SCAA3, SCAA4) as an example. When preparing the applications, the applicable and generally accepted pharmaceutical rules for the production of (compounded) drugs are applied (for example, European Pharmacopoeia, German Pharmaceutical Codex).

SCAA1: PC+PVP=1:3→mix→e.g., in a crimp top bottle→dissolve directly before use

100 mg of ProcCluster (carbonic acid adduct) and 300 mg of PVP25 (mass ratio 1:3) dissolved in 5 ml of water (Water for Injection) or 0.9% sodium chloride solution or Sterofundin ISO solution. PVP=polyvinylpyrrolidone, also called polyvidone or povidone This solution is sterile filtered using a sterile filter (e.g., PES), added to 100 ml of water (Water for Injection) or 0.9% sodium chloride solution or Sterofundin ISO solution and administered directly as an infusion solution.

The solid mixture is stable for a period of 6 months when stored in the refrigerator and at room temperature. The solution is stable for a period of 24 h after production.

SCAA1: carbonic acid adduct (CAA)+PVP=1:3 as a solution

2 g of carbonic acid adduct (CAA) and 6 g of PVP25 (mass ratio 1:3) dissolved in 200 ml of CO₂-saturated water (Water for Injection) and again saturated with CO₂. PVP=polyvinylpyrrolidone, also called polyvidone or povidone

This solution is sterile filtered and filled into crimp top bottles. The solution is stable for a period of 12 months after production.

The production of the carbonic acid adduct (CAA) is disclosed in WO2019048590 (A1).

SCAA2: (carbonic acid adduct (CAA)+NaHCO₃)+PVP=1:5

### 3.1 Materials:

Amine (AM): 48.93 g of procaine hydrochloride (e.g., ultra pure, for use as a pharmaceutical active ingredient; Ph. Eur. or in a quality suitable for this purpose)
Base (BA): 15.07 g of sodium bicarbonate (e.g., ultra pure, or in a quality suitable for this purpose),
22.5 g PVP 25
Solvent: 450 ml of water (Water for Injection)
CO₂: at least 7.5 g/l of carbon dioxide from pressurized steel cylinders (CO₂ of suitable quality)
Dry ice for the preparation of cooling mixtures and for cooling
Methanol, technical for the production of cooling mixtures

### 3.2 Step a)

Water (e.g., Water for Injection) is filled into a cleaned plastic pressure bottle up to the mark (approx. 400 ml) and pre-cooled for at least 1 h in the refrigerator (3 to 8° C.) or by means of external cooling to 5° C.

A carbonic acid solution saturated with carbon dioxide is prepared. For this purpose, CO₂ is introduced into the pre-cooled water at intervals under pressure (1.6 to 8 bar). The hissing (escaping gas through the pressure relief valve) indicates saturation of the solution with CO₂. The saturation is controlled by weight until 2.0 to 4.0 g of CO₂ (corresponding to 4.5 to 7.5 g/l) are dissolved. The saturated solution has a pH of ≤4.3 to 4.8. This carbonated water is immediately sealed and stored in the refrigerator for at least 1 h.

### 3.3 Step b)

In a second plastic pressure bottle, 15.07 g of sodium bicarbonate is placed, mixed with 200 ml of cooled CO₂-containing water and dissolved while swirling.

### 3.4 Step c)

To this solution, the equivalent amount of solid procaine hydrochloride is added at constant temperature, forming a nearly neutral solution which, after adding a further 200 ml of cold carbonated water, yields a clear, weakly acidic solution. The solution is enriched with CO₂. The solution prepared in this way is stored in the refrigerator for at least 1 h.

### 3.5 Step g2)

The PVP solution is prepared in a third plastic pressure bottle fitted with a magnetic stirrer. For this purpose, 200 ml of water (Water for Injection) are placed in the bottle and 22.5 g of PVP is added. The stirring rate is adjusted as needed. The complete dissolution is verified. The solution must be clear. To this solution, 28.2 ml of the previously prepared solution from step c) are added and this mixture is made up to 450 ml with water (Water for Injection). The solution is then conditioned again with CO₂.

This solution is sterile filtered and filled into crimp top bottles. The solution is stable for a period of 12 months after production.

SCAA3/SCAA4: produced from lyophilizate comprising galactose

10 g of stabilized carbonic acid adduct lyophilizate (SCAA3) or (SCAA4) comprising galactose (weight ratio carbonic acid adduct: PVP17:galactose=1:3:1) are dissolved in 200 ml of CO₂-saturated water (Water for Injection) and saturated again with CO₂. This solution is sterile filtered and filled into crimp top bottles. The solution is stable for a period of 12 months after production.

### 4. Decomposition Point

The decomposition points of the following carbonic acid adducts were determined.
F_{dec} carbonic acid adduct CAA without PVP addition based on procaine (production as described in the experimental part of WO2019048590 A2 by way of example): 65-85° C.
F_{dec} carbonic acid adduct SCAA3 (as prepared in the production example for SCAA3 above) based on procaine: 120-145° C.
F_{dec} carbonic acid adduct SCAA1 (carbonic acid adduct: polyvinylpyrrolidone 1:3 (w/w)): 70 to 90° C.

1. A stabilized carbonic acid adduct (SCAA1), comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
d) freezing the solution yielded after completion of step c),
e) storing the solution frozen in step d) at −100 to 0° C. for no longer than 4 days,
g1) adding polyvinylpyrrolidone to the carbonic acid adduct (CAA) yielded in the preceding steps to yield the stabilized carbonic acid adduct (SCAA1),
wherein the content of CO₂ in the solution which is subjected to step d) is at least 6 g/l, preferably at least 7 g/l, more preferably at least 8 g/l, particularly preferably 8.1 g/l to 9.5, and the amine (AM) can also be employed in the form of a salt.

2. The stabilized carbonic acid adduct (SCAA2), comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
g2) adding polyvinylpyrrolidone to the solution (B) yielded in step c) to yield the stabilized carbonic acid adduct (SCAA2).
wherein the content of CO₂ in the solution (B) which is subjected to step c) is at least 10 g/l, preferably at least 12 g/l, more preferably at least 14 g/l, and most preferably at least 14 to 16 g/l, and the amine (AM) can also be employed in the form of a salt.

3. The stabilized carbonic acid adduct (SCAA3) comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
g3) adding polyvinylpyrrolidone to the solution (B) yielded in step c) to yield solution (C),
h) freezing the solution yielded after completion of step g3),
j) storing the solution frozen in step h) at −100 to 0° C. for no longer than 4 days, wherein the CO₂ content in the solution subjected to step h) is at least 7 g/l, preferably at least 7.5 g/l, and the amine (AM) can also be employed in the form of a salt,
wherein the steps are carried out in the order listed above: a), optionally b), c), g3), h) and j).

4. The stabilized carbonic acid adduct (SCAA4) comprising carbonic acid, at least one amine (AM) and, optionally, at least one salt (S),
producible according to a method comprising the steps of:
a) providing a solution (A) comprising at least one solvent and CO₂ dissolved in the at least one solvent,
optionally, b) dissolving a base (BA) other than the amine (AM) in the solution (A) to yield the solution (A1),
c) dissolving the at least one amine (AM) in the solution (A) or (A1) to yield the solution (B),
d) freezing the solution yielded after completion of step c),
e) storing the solution frozen in step d) at −100 to 0° C. for no longer than 4 days,
f) drying the solution stored in step e) to yield carbonic acid adduct (CAA),
k) drying a solution saturated with CO₂ comprising the carbonic acid adduct (CAA) from step f) and polyvinylpyrrolidone,
wherein the CO₂ content in the solution subjected to step d) is at least 6 g/l, preferably at least 7 g/l, more preferably at least 8 g/l, particularly preferably 8.1 g/l to 9.5 g/l and the amine (AM) can also be employed in the form of a salt.

5. A kit, comprising
a) carbonic acid adduct (CAA) produced as defined in claim 1;
b) polyvinylpyrrolidone;
optionally, c): an isotonic solution or non-isotonic solution, preferably an isotonic solution, preferably selected from 0.9% (w/w) NaCl solution, or a full electrolyte solution, preferably selected from Stereofundin, Stereofundin ISO, Ringer's solution, Ringer's acetate, Ringer's lactate, Deltajonin, Jonosteril, and Jonosteril malate,
wherein the carbonic acid adduct (CAA) and the polyvinylpyrrolidone are optionally present as a solid mixture.

6. The stabilized carbonic acid adduct (SCAA1), (SCAA2), (SCAA3) or (SCAA4) according to any one of claims 1 to 4, and the carbonic acid adduct (CAA) in claim 5 according to claim 1, wherein step a) comprises at least one of the following substeps:
a1) cooling the solvent, preferably water, to 3 to 8° C., preferably 5° C. and/or
a2) introducing CO₂ into the solvent, preferably up to a saturation concentration of 3 to 10 g/l, more preferably up to a saturation concentration of 4.5 to 7.5 g/l, preferably the pH of the solution after saturation with CO₂ is ≤3.0 to 6.0, even more preferably ≤4.3 to 4.8, and/or
a3) storing the solution (A) at 1 to 10° C., preferably for at least 30 min, more preferably for at least 50 min, even more preferably for at least 60 min; up to a maximum of 5 days (120 h); preferably, storing is carried out at 3 to 8° C., preferably for at least 30 min, more preferably for at least 50 min, even more preferably for at least 60 min; up to a maximum of 5 days (120 h);
preferably, step a) comprises all substeps a1), a2) and a3),
preferably, the substeps a1), a2) and a3) are carried out in the order a2) follows a1) and a3) follows a2).

7. The stabilized carbonic acid adduct (SCAA1), (SCAA2), (SCAA3) or (SCAA4) according to any one of claims 1 to 4, 6 and the carbonic acid adduct (CAA) in claim 5 according to claim 1, wherein the base (BA) in step b) is a bicarbonate or a carbonate, more preferably a bicarbonate, even more preferably sodium bicarbonate.

8. The stabilized carbonic acid adduct (SCAA1), (SCAA2), (SCAA3) or (SCAA4) according to any one of claims 1 to 4, 6, 7 and the carbonic acid adduct (CAA) in claim 5 according to claim 1, wherein step c) comprises at least one of the following substeps:
c1) dissolving the at least one amine (AM) in the solution (A) or (A1) to yield the solution (B), and/or
c2) adding solution (A) to solution (B) to yield solution (B1), and/or
c3) enriching solution (B) or (B1) with CO₂, and/or
c4) storing the solution (B) or (B1) at 1 to 10° C., preferably 3 to 8° C., for at least 1 h, preferably 24 h to 120 h, even more preferably 24 to 72 h, and/or
c5) enriching the solution (B) or (B1) with CO₂ to a concentration of at least 6 g/l, preferably at least 10 g/l, more preferably at least 12 g/l, even more preferably at least 14 g/l, and most preferably at least 15 g/l;
wherein, optionally,
i) the concentration of the amine (AM) in solution (B) or, when carrying out substep c2), in solution (B1) is 0.01 to 0.25 g/ml, preferably 0.03 to 0.20 g/ml, more preferably 0.08 to 0.15 g/ml, and/or
ii) the pH of solution (B) or (B1) after carrying out step c5) is ≤7.0, and/or
iii) the ratio of the amine (AM) to the base (BA), when carrying out step b), in solution (B) is 2:1 to 5:1, more preferably 3:1 to 4:1, even more preferably 3.23:1 to 3.26:1 [g/g], and/or
iv) in step c1), the at least one amine (AM) comprises the at least one amine (AM) as an acid addition salt, preferably as hydrohalide, bisulfate, bisulfite, biphosphate, hydromesylate, hydrotosylate, hydroacetate, hydroformate, hydropropanoate, hydromalonate, hydrosuccinate, hydrofumarate, hydroxalate, hydrotartrate, hydrocitrate, hydromaleate, more preferably as hydrochloride or hydrobromide,
preferably, step c) comprises all substeps c1), c2), c3), c4) and c5);
preferably, substeps c1), c2), c3), c4) and c5) are carried out in the order c2) follows c1), c3) follows c2), c4) follows c3), c5) follows c4).

9. The stabilized carbonic acid adduct (SCAA1) or (SCAA4) according to any one of claims 1 to 4, 6 to 8, and the carbonic acid adduct (CAA) in claim 5 according to claim 1, wherein in step d):
i) the solution (B) or (B1) is frozen at −100° C. to −20° C., preferably at −90° C. to −30° C., more preferably at −80 to −40° C., and most preferably at −70 to −50° C., and/or
ii) the solution (B) or (B1) is frozen within 0.3 to 60 minutes, preferably within 1 to 30 minutes, more preferably within 1.1 to 10 minutes, even more preferably within 1.5 to 5 minutes, and/or
iii) the vessel in which the solution (B) or (B1) is located during the freezing process, preferably in the cooling medium, is rotated at 10 to 1000 rpm, preferably at 50 to 600 rpm, more preferably at 100 to 400 rpm, and even more preferably at 200 to 300 rpm, and/or
iv) the solution (B) or (B1) is frozen at a cooling rate of 10 to 100 K/min, preferably 20 to 80 K/min, more preferably 30 to 70 K/min, and particularly preferably 40 to 60 K/min.

10. The stabilized carbonic acid adduct (SCAA1) or (SCAA4) according to any one of claims 1 to 4, 6 to 9, and the carbonic acid adduct (CAA) in claim 5 according to claim 1, wherein in step e):
i) the frozen solution (B) or (B1) is stored for 1.5 to 4 days, preferably for 2.5 to 4 days, and/or
ii) the frozen solution (B) or (B1) is preferably stored at −50 to 0° C., more preferably at −30 to −5° C., even more preferably at −25 to ~10° C., particularly preferably at −20 to ~15° C.

11. The carbonic acid adduct (SCAA1), (SCAA3) or (SCAA4) according to any one of claims 1 to 4, 6 to 10, and the carbonic acid adduct (CAA) in claim 5 according to claim 1, wherein the method comprises a further step f) which is carried out after step e) or j),
f) drying the solution stored in step e) or j) to yield dried carbonic acid adduct (SCAA1) or (SCAA3); or (CAA)
wherein, in step f), optionally,
i) the water is removed from the solution (B) or (B1) to a residual content of <0.8 wt. %, preferably <0.1 wt. %, based on the total weight of the drying product (C), and/or
ii) CO₂ not bound in the carbonic acid adduct (CAA) is removed from the solution (B) or (B1) to a residual content of <0.8 wt. %, preferably <0.1 wt. %, based on the total weight of the drying product (C), and/or
iii) drying is carried out by lyophilization, and/or
iv) the pressure during drying is 0.01 to 30 mbar, preferably 0.02 to 20 mbar, more preferably 0.03 to 10 mbar, even more preferably 0.03 to 0.5 mbar, and most preferably 0.05 to 0.1 mbar, and is preferably maintained throughout the entire drying process, and/or
v) the pressure during drying according to iv) is reached within 10 h, preferably within 7 h, more preferably within 5 h, and particularly preferably within 4 h from the start of evacuation, and/or
vi) the temperature during the entire drying in step f) is 0 to 20° C., preferably 4 to 18° C., more preferably 8 to 16° C., and/or
vii) the total drying time is 10 to 60 h, preferably 30 to 55 h, more preferably 41 to 52 h.

12. The stabilized carbonic acid adducts (SCAA1), (SCAA2), (SCAA3) and (SCAA4) according to any one of claims 1 to 4, 6 to 11, and the carbonic acid adduct (CAA) in claim 5 according to claim 1, wherein
A) at least one amine (AM) according to any one of the following formulas (I) or (II) is employed, 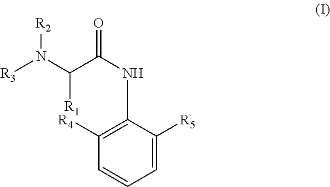
wherein, in formula (I),
R₁ is H, C₁₋₁₀ alkyl, C₂₋₁₀ alkenyl, aryl or heteroaryl, preferably H or C₁₋₁₀ alkyl, more preferably H or C₁₋₄ alkyl;
R₂ is H, C₁₋₁₀ alkyl, C₂₋₁₀ alkenyl, aryl or heteroaryl, preferably H or C₁₋₁₀ alkyl, more preferably H or C₁₋₄ alkyl;
R₃ is H, C₁₋₁₀ alkyl, C₂₋₁₀ alkenyl, aryl or heteroaryl, preferably H or C₁₋₁₀ alkyl, more preferably H or C₁₋₄ alkyl;
wherein, optionally, R₁ and R₃ may be linked together, and together with the nitrogen atom bonded to R₃ and the carbon atom bonded to R₁ may form a saturated or unsaturated, preferably a saturated, ring, preferably, the ring is a 4, 5 or 6 membered ring, more preferably a 5 or 6 membered ring,
R₄ is H, halo, C₁₋₁₀ alkyl, C₂₋₁₀ alkenyl, aryl or heteroaryl, preferably H or methyl;
R₅ is H, halo, C₁₋₁₀ alkyl, C₂₋₁₀ alkenyl, aryl or heteroaryl, preferably H, methyl or halo; 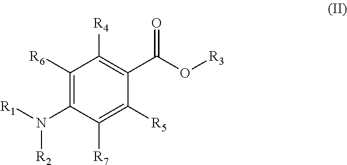
wherein, in formula (II),
R₁ is H, C₁₋₁₀ alkyl, C₂₋₁₀ alkenyl, aryl or heteroaryl, preferably H or C₁₋₁₀ alkyl, more preferably H;
R₂ is H, C₁₋₁₀ alkyl, C₂₋₁₀ alkenyl, aryl or heteroaryl, preferably H or C₁₋₁₀ alkyl, more preferably H;
R₃ is H, C₁₋₁₀ alkyl, C₂₋₁₀ alkenyl, aryl, heteroaryl or —(CH₂)ₙNR₈R₉, preferably —(CH₂)ₙNR₈R₉,
n is 1 to 5, preferably 1 to 3, more preferably 1 to 2;
R₈ is C₁₋₁₀ alkyl, preferably C₁₋₂ alkyl;
R₉ is C₁₋₁₀ alkyl, preferably C₁₋₂ alkyl;
R₄ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or halo;
R₅ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or halo;
R₆ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or —O—C₁₋₁₀ alkyl;
R₇ is H, halo, C₁₋₁₀ alkyl, C₂₋₁₀ alkenyl, aryl, heteroaryl or —O—C₁₋₁₀ alkyl, preferably H, halo, C₁₋₁₀ alkyl or —O—C₁₋₁₀ alkyl, more preferably H or —O—C₁₋₁₀ alkyl,
wherein the at least one amine according to formula (I) and (II) can, optionally, also be employed in the form of a salt, or
B) at least one amine (AM) is employed, selected from the group 4-aminobenzoic acid 2-(N,N-diethylamino)ethyl ester (procaine), 4-aminobenzoic acid ethyl ester (benzocaine), 2-(diethylamino)ethyl 4-amino-2-chlorobenzoate (chloroprocaine), 4-amino-3-butoxybenzoic acid 2-diethylaminoethyl ester (oxybuprocaine), (2-(dimethylamino)ethyl)-4-(butylamino)benzoate (tetracaine), N-[3-(4-phenoxymethylphenyl) propyl]morpholine (fomocaine), 2-diethylamino-N-(2,6-dimethylphenyl) acetamide (lidocaine), (RS)—N-(2,6-dimethylphenyl)-1-methylpiperidine-2-carboxamide (mepivacaine), (RS)—N-(2-methylphenyl)-2-(propylamino) propanamide (prilocaine), (RS)-4-methyl-3-[2-(propylamino) propanamido]thiophene-2-carboxylic acid methyl ester (articaine), (±)-1-butyl-N-(2,6-dimethylphenyl)-2-piperidinecarboxamide (bupivacaine), (S)-1-propyl-2′,6′-dimethyl-2-piperidylcarboxyanilide (ropivacaine), 2-(ethylpropylamino)-2′,6′-butyroxylidide (etidocaine), 1-(4-butoxyphenyl)-3-piperidin-1-ylpropan-1-one (dyclonine), preferably 4-aminobenzoic acid-2-(N,N-diethylamino)ethyl ester (procaine) and 2-diethylamino-N-(2,6-dimethylphenyl) acetamide (lidocaine) (2-(dimethylamino)ethyl)-4-(butylamino)benzoate (tetracaine) particularly preferably 4-aminobenzoic acid 2-(N,N-diethylamino)ethyl ester (procaine) and/or the salts of these compounds.

13. Production method of
i) the stabilized carbonic acid adduct (SCAA1) according to any one of claims 1 and 6 to 12, comprising the steps according to any one of claims 1 and 6 to 12, based on the stabilized carbonic acid adduct (SCAA1); or
ii) the stabilized carbonic acid adduct (SCAA2) according to any one of claims 2 and 6 to 12, comprising the steps according to any one of claims 2 and 6 to 12, based on the stabilized carbonic acid adduct (SCAA2); or
iii) the stabilized carbonic acid adduct (SCAA3) according to any one of claims 3 and 6 to 12, comprising the steps according to any one of claims 3 and 6 to 12, based on the stabilized carbonic acid adduct (SCAA3); or
iv) the stabilized carbonic acid adduct (SCAA4) according to any one of claims 4 and 6 to 12, comprising the steps according to any one of claims 4 and 6 to 12, based on the stabilized carbonic acid adduct (SCAA4).

14. The stabilized carbonic acid adducts (SCAA1), (SCAA2), (SCAA3) and (SCAA4) according to any one of claims 1 to 4 and 6 to 12 for use in a method of anesthesia, of analgesia, of concomitant treatment of cancer, of treatment of amyotrophic lateral sclerosis (ALS), of anti-inflammatory treatment, of supporting wound healing, in particular in burns, open wounds and scars, of treatment of neurogenic inflammations such as multiple sclerosis, MMN (multifocal motor neuropathy), of treatment of sinusitis, of treatment of asthma, of treatment of rheumatoid arthritis, of treatment of Alzheimer's disease, of treatment of dementia, of supporting convalescence and of supporting anti-aging, of treatment of burnout syndrome, of treatment of osteoarthritis, of treatment of polyarthritis, of treatment of pain syndrome and general pain, of pre- and post-operative treatment (also for bone fractures), of preventive and rehabilitation medicine, of treatment of zoster neuralgia, of accompanying treatment of zoster neuralgia, of treatment of diseases of the abdominal organs such as liver, gallbladder, pancreas, intestines, of treatment of gastrointestinal diseases (ulcerative colitis, Crohn's disease), of treatment of ankylosing spondylitis, of treatment of chronic musculoskeletal pain, of treatment of diabetes (improvement of blood sugar levels), of reducing edema, of comedication to opioids.

15. The stabilized carbonic acid adduct (SCAA1) according to any one of claims 1, 4 and 6 to 14, or the kit according to claim 5, wherein
i) in step g1) or in the solid mixture the weight ratio of polyvinylpyrrolidone (PVP) to carbonic acid adduct (CAA) is 10:1, more preferably 5:1, particularly preferably 3:1; and/or
ii) the polyvinylpyrrolidone (PVP) has an average molecular weight of 10,000 to 40,000 g/mol, preferably of 15,000 to 35,000 g/mol, more preferably of 24,000 to 33,000 g/mol, particularly preferably of 17,000 g/mol to 32,000 g/mol; and/or
iii) the administration is parenteral, buccal, by inhalation, oral, sublingual or lingual.

16. The stabilized carbonic acid adduct (SCAA2) according to any one of claims 2 and 6 to 14, wherein,
i) in step g2), the weight ratio of polyvinylpyrrolidone (PVP) to carbonic acid adduct (CAA) is preferably 20:1, more preferably 10:1, particularly preferably 5:1; and/or
ii) the polyvinylpyrrolidone (PVP) has an average molecular weight of 15,000 to 35,000 g/mol, more preferably of 24,000 to 33,000 g/mol, and particularly preferably of 16,000 g/mol to 27,000 g/mol; and/or
iii) the administration is buccal, by inhalation, parenteral, oral, lingual or sublingual.

17. The stabilized carbonic acid adduct (SCAA3) according to any one of claims 3 and 6 to 14, wherein,
i) in step g3), the weight ratio of polyvinylpyrrolidone (PVP) to carbonic acid adduct (CAA) is preferably 6:1 to 1:0.5, more preferably 3:1, particularly preferably 1.5:1; and/or
ii) the polyvinylpyrrolidone (PVP) has an average molecular weight of 10,000 to 95,000 g/mol, preferably of 15,000 to 35,000 g/mol, more preferably of 24,000 to 33,000 g/mol, particularly preferably of 17,000 g/mol to 32,000 g/mol; and/or
iii) the administration is parenteral, buccal, by inhalation, oral, sublingual or lingual, and/or
iv) the stabilized carbonic acid adduct (SCAA3) is formulated in tablet form, preferably when using polyvinylpyrrolidone (PVP) in step g3) with an average molecular weight of 24,000 to 33,000 g/mol, more preferably from 17,000 g/mol to 30,000 g/mol, and/or
v) the stabilized carbonic acid adduct (SCAA3) is formulated as an ointment with a water-based base; and/or
vi) the stabilized carbonic acid adduct (SCAA3) is formulated as a gel; and/or
vii) in step g3), one or more further additives are added, selected from a monosaccharide, preferably galactose and glucose, and/or
viii) the administration is parenteral, buccal, by inhalation, oral, lingual or sublingual.

18. The stabilized carbonic acid adduct (SCAA4) according to any one of claims 4 and 6 to 14, wherein,
i) in step k), the weight ratio of polyvinylpyrrolidone (PVP) to carbonic acid adduct (CAA) is preferably 6:1 to 1:0.5, more preferably 3:1, particularly preferably 1.5:1; most preferably 1:0.5; and/or
ii) the polyvinylpyrrolidone (PVP) has an average molecular weight of 10,000 to 95,000 g/mol, preferably of 15,000 to 35,000 g/mol, more preferably of 24,000 to 33,000 g/mol, particularly preferably of 17,000 g/mol to 32,000 g/mol; and/or
iii) the administration is parenteral, buccal, by inhalation, oral, lingual or sublingual; and/or
iv) the stabilized carbonic acid adduct (SCAA4) is formulated as a gel.
